(19) United States
(12) Patent Application Publication
(10) Pub. No.: US 2026/0000367 A1
(43) Pub. Date: Jan. 1, 2026

(54) PATIENT SUPPORT APPARATUS MONITORING WITH ALERT REDUCTION

(71) Applicant: UMANO MEDICAL INC., L'Islet (CA)

(72) Inventors: Caroline GRIMARD, St-Henri (CA); Julien THIBOUTOT, Sherbrooke (CA); Tristan DUMAIS, Quebec (CA); David MAYEN MORENO, L'Ancienne-Lorette (CA); Steve BOLDUC, Beaumont (CA)

(73) Assignee: UMANO MEDICAL INC., L'Islet (CA)

(21) Appl. No.: 19/317,956

(22) Filed: Sep. 3, 2025

Related U.S. Application Data
(63) Continuation-in-part of application No. 15/818,248, filed on Nov. 20, 2017, now Pat. No. 10,497,247.
(60) Provisional application No. 63/778,794, filed on Mar. 27, 2025.

(51) Int. Cl. A61B 5/00; A61B 5/11; A61G 7/05

(52) U.S. Cl. CPC A61B 5/746; A61B 5/1115; A61G 7/0527; A61B 2562/0252; A61G 2203/20

(57) ABSTRACT

A patient support apparatus, a monitoring system and a monitoring method comprising providing a state of a component of the patient support apparatus, receiving at least one input indicative of a start command to activate monitoring of the component and/or a pause command to pause outputting of an alert indicator for the activated monitoring of the component, providing at least one output indicative of the alert indicator, determining if at least one initial condition has occurred including a presence of the patient on the patient support apparatus, determining if the alarm condition has occurred based on the input, the alarm condition and the state of the component, and providing the alert indicator if the alarm condition has occurred, in the presence of the initial condition, in the presence of the start command and in an absence of the pause command.

### CROSS-REFERENCE TO RELATED APPLICATIONS

The present application claims priority to U.S. Provisional Patent Application Ser. No. 63/778,794 filed on Mar. 27, 2025 and is a Continuation-in-Part of U.S. patent application Ser. No. 15/818,248 filed on Nov. 20, 2017, the entirety of both being hereby incorporated by reference.

### TECHNICAL FIELD

The present generally relates to monitoring of systems and components of a patient support apparatus, such as a hospital bed. More particularly, it concerns the reduction of the number of alert indicators outputted by monitoring systems to reduce alarm fatigue among caregivers.

### BACKGROUND

Healthcare continues to become increasingly computerized, and caregivers use an assortment of equipment and technology to monitor patient conditions. Most healthcare devices provide auditory or visual warnings intended to alert caregivers when a patient's condition or environment deviates from a predetermined normal value or range. Many device alerts emit different sounds, tones, and/or pitches depending on the level of severity (i.e., advisory vs. warning vs. crisis alerts) to help caregivers determine how to respond. System status or non-clinical alerts or notifications can also occur and can be caused by mechanical or electrical problems or by physical states of the equipment.

Alarm fatigue occurs when caregivers experience high exposure to medical device alerts, causing alarm desensitization and leading to missed alerts or delayed response. As the frequency of alerts used in healthcare rises, alarm fatigue has been increasingly recognized as an important patient safety issue. Alarm desensitization is compounded by the fact that false or nonactionable alerts occur frequently. False alarms are those that occur in the absence of an intended valid event, and nonactionable alerts occur when a monitoring system works as designed but signifies an event that is not clinically significant and/or requires no additional intervention. The high volume of these nuisance alerts is not only disruptive, but also creates a situation where staff doubt the reliability of alerts and as a result turn down the volume, ignore, or deactivate the alerts. This adversely affects patient safety because caregivers are not only ignoring nuisance alerts but also ignoring or missing many clinically significant and actionable alerts.

In a hospital bed equipped with either a simple or a technical mattress, a plurality of monitoring functions can be provided for the complex assortment of systems and components. They are sometimes referred to as bed status monitoring.

Some clinical conditions require specific hospital bed configurations, including patients with cardiovascular conditions, respiratory issues, mobility issues, pain relief management, pressure ulcer prevention, circulation issues, cognitive impairments, incontinence management, fall risk, weight management, etc. The hospital bed configuration that is recommended for these patients may include values or ranges for the elevation of the bed from the floor (hi-low adjustment), the length of the bed, the width of the bed, the angles of the head, back, seat, thigh, leg and foot rests, the position of the siderails, the tilt of the deck for foot or head elevation (Trendelenburg and Reverse Trendelenburg Adjustment), the tilt of the deck for left or right side elevation (lateral tilting), the head-of-bed (HOB) angle, the detected weight on the bed, the inflation status of each zone of the mattress, the activation of specific functions of the mattress, the level of humidity under the patient, etc.

Bed status monitoring is designed to detect the current state of the bed and to compare it with the pre-determined required configuration for the patient. Bed status alerts are emitted when the current state of the bed departs from the preset or desired configuration for the patient.

One special type of bed status monitoring is bed exit monitoring. Most falls in nursing homes occur in the resident's room, especially during attempts to get in or out of bed. Bed exit monitoring, designed to detect patient's movement out of bed, increase staff surveillance of cognitively and/or physically impaired residents at risk for bed-related falls. Although bed exit monitoring can be done using components directly attached to the resident such as a garment clip or position change alert, when part of a bed status monitoring, they are usually provided as part of the bed itself (e.g., pressure-sensitive mats, bedside infrared beam detectors, or in-bed pressure sensors). Bed exit alerts emitted as part of bed exit monitoring inform staff when a person is lifting their head and upper torso, attempting to leave the bed by moving to one of its sides, has gotten out of bed, or has fallen out of the bed. This is typically done by detecting a movement of the patient on the bed, by determining their center of mass on the surface or by using pressure sensors. Bed exit monitoring therefore involves monitoring the presence of a patient on the bed and the change in their position or presence on the bed.

When caregivers enter a patient's room to provide care to the patient, the first step they usually take is to stop all bed status monitoring to avoid triggering alerts during care. In some prior art systems, the caregiver must separately stop the bed status monitoring and the bed exit monitoring. Once the care is completed or when a higher priority task is required, the caregiver needs to manually reactivate bed status and bed exit monitoring. If some time has elapsed or if the caregiver is preoccupied or tired, they may forget to reactivate the monitoring or forget exactly which monitoring was previously set up.

The caregiver may prefer to leave the monitoring active and armed with all settings unchanged during the care to avoid forgetting to arm them again or entering incorrect settings after the care is provided. This may lead to further alerts being triggered by the bed status monitoring as the caregiver manipulates the patient and the bed components to care for the patient.

To alleviate alarm fatigue, when the bed status monitoring expects the patient to be in bed and the siderails to be in the raised position, for example, the caregiver may prefer to lower the siderail, ignore or disregard the siderail monitoring alert, help the patient exit the bed and raise the siderail to stop the alert. This leads to additional steps to be taken care of by the caregiver to lower the siderails before being able to let a patient back in bed. Additionally, the caregiver may need to let go of the patient to move the siderails or may need to help a patient to a chair before being able to lower the siderails and then help the patient back to the bed. These extra manipulations of the patient and the equipment disrupt the natural workflow and increase safety risks.

There is therefore a need for a system and method for hospital bed monitoring which contributes to reducing alarm fatigue among caregivers by reducing the number of generated alerts.

### SUMMARY

It is an object of the present to improve at least some of the inconveniences present in the prior art.

In accordance with a broad aspect of the present disclosure, there is provided a monitoring system for a patient support apparatus, the monitoring system comprising: a sensor assembly operatively connected to the patient support apparatus, the sensor assembly being operable to provide an indication of a state of a component of the patient support apparatus, a user interface operable to receive at least one input and provide at least one output, the input being indicative of at least one of: a start command to activate monitoring of the component and a pause command to pause outputting an alert indicator for the activated monitoring of the component, the output being indicative of the alert indicator, a memory operable to store computer-readable instructions including instructions indicative of an alarm condition for the state of the component, and a processing unit operatively connected to the sensor assembly, the user interface and the memory, the processing unit being operable to: receive, from the user interface, the at least one input, determine if at least one initial condition has occurred, the at least one initial condition includes a presence of the patient on the patient support apparatus, determine if the alarm condition has occurred based on the input and the state of the component, and provide the alert indicator if the alarm condition has occurred, in the presence of the initial condition, in the presence of the start command and in an absence of the pause command.

In one or more embodiments, the processing unit is operable to: determine if the state of the component is related to one of a bed exit of the patient and a bed status of the patient support apparatus, determine if the patient is currently present on the patient support apparatus, and provide the alert indicator if the state of the component is related to the bed status and if the patient is currently present on the patient support apparatus.

In one or more embodiments, the monitoring system further comprises at least one of: a light assembly, an audio output assembly, a display and an application operatively connected to the processing unit to provide the alert indicator.

In one or more embodiments, the user interface comprises at least one of: a touchscreen and a control element configured to receive the input from the user.

In one or more embodiments, the memory comprises storage for a log, and the processing unit is further operable to log at least one of: an indication of an occurrence of the start command, an indication of an occurrence of the pause command and an indication of the determination that the alarm condition has occurred.

In one or more embodiments, the input is indicative of a desired state for the component and the processing unit is operable to use the desired state for the determining if the alarm condition has occurred.

In one or more embodiments, the component comprises at least one of: a siderail wherein the desired state is the siderail being raised, a brake wherein the desired state is the brake being set, an elevation system wherein the desired state is one of a height of the patient support apparatus and an angle of a tilt of the patient support apparatus, and a panel wherein the desired state is an angle of the panel.

In one or more embodiments, the sensor assembly comprises at least one load cell configured for determining a weight distribution of the patient on the patient support apparatus and a presence of the patient on the patient support apparatus.

In one or more embodiments, if the input is the pause command, the processing unit is further configured for: displaying a time remaining in the pause on the user interface, displaying a pause extension on the user interface to extend the pause, and in response to the time remaining expiring and the user not selecting the pause extension: providing the alert indicator.

In accordance with a broad aspect of the present disclosure, there is provided a patient support apparatus comprising: a frame, a patient receiving surface supported by the frame, a sensor assembly operable to transmit an indication of a state of a component of the patient support apparatus, a user interface operable to receive at least one input and provide at least one output, the input being indicative of at least one of a start command to activate monitoring of the component and a pause command to pause outputting an alert indicator for the activated monitoring of the component, and the output being indicative of the alert indicator, a memory operable to store computer-readable instructions including instructions indicative of an alarm condition for the state of the component, and a processing unit operatively connected to the sensor assembly, the user interface and the memory, the processing unit being operable to: determine if at least one initial condition has occurred, the at least one initial condition includes a presence of the patient on the patient receiving surface, determine if the alarm condition has occurred based on the input and the state of the component, and provide the alert indicator if the alarm condition has occurred, in the presence of the initial condition, in the presence of the start command and in an absence of the pause command.

In one or more embodiments, the processing unit is operable to: determine if the state of the component is related to one of a bed exit of the patient and a bed status of the patient support apparatus, determine if the patient is currently present on the patient support apparatus, and provide the alert indicator if the state of the component is related to the bed status and if the patient is currently present on the patient support apparatus.

In one or more embodiments, the input is indicative of a desired state for the component and the processing unit is operable to use the desired state for the determining if the alarm condition has occurred.

In one or more embodiments, the component comprises at least one of: a siderail the desired state is the siderail being raised, a brake the desired state is the brake being set, an elevation system the desired state is one of a height of the patient support apparatus and an angle of a tilt of the patient support apparatus, and a panel in the desired state is an angle of the panel.

In accordance with a broad aspect of the present disclosure, there is provided a monitoring method for a patient support apparatus, the monitoring method being executed by at least one processing unit operatively connected to a patient support apparatus and at least one user interface for receiving at least one input and providing at least one input, the monitoring method comprising: determining a state of a component of the patient support apparatus, receiving at least one input, the input being indicative of at least one of a start command to activate monitoring of the component and a pause command to pause outputting an alert indicator for the activated monitoring of the component, providing at least one output, the output being indicative of the alert indicator, retrieving instructions indicative of an alarm condition for the state of the component, determining if at least one initial condition has occurred, the at least one initial condition includes a presence of the patient on the patient support apparatus, determining if the alarm condition has occurred based on the input, the instructions and the state of the component, and providing the alert indicator if the alarm condition has occurred, in the presence of the initial condition, in the presence of the start command and in an absence of the pause command.

In one or more embodiments, the monitoring method further comprises: determining if the state of the component is related to one of a bed exit of the patient and a bed status of the patient support apparatus, determining if the patient is currently present on the patient support apparatus, and the providing the alert indicator is dependent on if the state of the component is related to the bed status and if the patient is currently present on the patient support apparatus.

In one or more embodiments, the input is indicative of a desired state for the component and the method comprises determining if the alarm condition has occurred based on the desired state.

In one or more embodiments, the component comprises at least one of: a siderail wherein the desired state is the siderail being raised, a brake wherein the desired state is the brake being set, an elevation system wherein the desired state is one of a height of the patient support apparatus and an angle of a tilt of the patient support apparatus, and a panel wherein the desired state is an angle of the panel.

In one or more embodiments, if the input is the pause command, the method further comprises: displaying a time remaining in the pause on the user interface, displaying a pause extension on the user interface to extend the pause, and in response to the time remaining expiring and the user not selecting the pause extension: providing the alert indicator.

In one or more embodiments, the monitoring method further comprises: storing, in a log, at least one of: an indication of an occurrence of the start command, an indication of an occurrence of the pause command and an indication of the determination that the alarm condition has occurred.

In one or more embodiments, the user interface comprises a touch screen.

In accordance with another broad aspect of the present disclosure, there is provided a patient support apparatus, a monitoring system and a monitoring method comprising determining a state of a component of the patient support apparatus, receiving at least one input indicative of a start command to activate monitoring of the component and/or a pause command to pause outputting of an alert indicator for the activated monitoring of the component, providing at least one output indicative of the alert indicator, retrieving an alarm condition for the state of the component, determining if at least one initial condition has occurred including a presence of the patient on the patient support apparatus, determining if the alarm condition has occurred based on the input, the alarm condition and the state of the component, and providing the alert indicator if the alarm condition has occurred, in the presence of the initial condition, in the presence of the start command and in an absence of the pause command.

Embodiments of the present technology each have at least one of the above-mentioned objects and/or aspects, but do not necessarily have all of them. It should be understood that some aspects of the present technology that have resulted from attempting to attain the above-mentioned objects may not satisfy these objects and/or may satisfy other objects not specifically recited herein.

Additional and/or alternative features, aspects, and advantages of embodiments of the present technology will become apparent from the following description, the accompanying drawings, and the appended claims.

### BRIEF DESCRIPTION OF THE DRAWINGS

Reference will now be made to the accompanying drawings, showing by way of illustration example embodiments thereof and in which:

FIG. 1 is a perspective view, taken from a top, front, left side, of a hospital bed according to an embodiment of the present technology;

FIG. 2 is a perspective view, taken from a top, front, right side, of a patient support assembly of the bed of FIG. 1, showing an upper body deck section thereof in a lowered position;

FIG. 3 is a side elevation view of the patient support assembly of FIG. 2;

FIG. 4 is a bottom plan view of the patient support assembly of FIG. 2;

FIG. 5 is a perspective view, taken from a top, front, right side, of the patient support assembly of FIG. 2, showing the upper body deck section in a raised position;

FIG. 6 is a perspective view, taken from a top, front, right side, of a patient support assembly according to another embodiment, showing the deck in a chair and the elevation system in a high configuration;

FIG. 7 is a block diagram of example components of a patient support apparatus;

FIG. 8 is a block diagram of example components of a communication system for a patient support apparatus;

FIG. 9 is a block diagram of example components of a monitoring system for a patient support apparatus;

FIG. 10 is a flow chart of example steps for a monitoring method;

FIG. 11 is a photograph of a patient support apparatus equipped with a patient support surface in a patient room environment;

FIG. 12 is a photograph of a patient installed on a patient support apparatus equipped with a patient support surface and linens in a patient room environment;

FIG. 13 is a graphical representation of a membrane and screen in one embodiment of a user interface for a endboard;

FIG. 14 is a graphical representation of a membrane in one embodiment of a user interface for a siderail;

FIG. 15 is a screenshot of a home page for a screen in another embodiment of a user interface;

FIG. 16 is a screenshot of a page for activating bed status monitoring in an example embodiment;

FIG. 17 is a screenshot of a page for activating bed exit monitoring in an example embodiment;

FIG. 18 includes FIG. 18A and FIG. 18B in which FIG. 18A shows a screenshot of a page shown during preparation mode and FIG. 18B shows a screenshot of a message shown during preparation mode, in an example embodiment;

FIG. 19 includes FIG. 19A, FIG. 19B and FIG. 19C in which FIG. 19A shows a screenshot of a home page where a Care Pause button is displayed, FIG. 19B shows a screenshot of a home page when a Care Pause has been commanded and FIG. 19C shows a screenshot of a Care Pause menu in an example embodiment;

FIG. 20 includes FIG. 20A, FIG. 20B, FIG. 20C and FIG. 20D in which FIG. 20A shows a screenshot of a home page where a Stop Alert/Care Pause button is displayed when a patient is present in the bed, FIG. 20B shows a screenshot of a home page when a Stop Alert/Care Pause has been commanded, FIG. 20C shows a screenshot of a home page where a Stop Alert button is displayed when a patient is not present in the bed and FIG. 20D shows a screenshot of a home page when a Stop Alert has been commanded in an example embodiment;

FIG. 21 includes FIG. 21A, FIG. 21B, FIG. 21C and FIG. 21D in which FIG. 21A shows a screenshot of a home page when the bed exit monitoring is disarmed, FIG. 21B shows a screenshot of a home page where bed exit monitoring is armed with auto-arm, FIG. 21C shows a screenshot of a home page where bed exit monitoring is armed and FIG. 21D shows a screenshot of a home page when a Stop Alert button is displayed because the bed exit is in alert in an example embodiment;

FIG. 22 includes FIG. 22A and FIG. 22B in which FIG. 22A shows a screenshot of a home page where there is a bed status alert and FIG. 22B shows a screenshot of a detail screen showing the cause of the alert, in an example embodiment;

FIG. 23 includes FIG. 23A and FIG. 23B in which FIG. 23A shows a screenshot of a home page where bed status and bed exit are armed and FIG. 23B shows a screenshot of a home page where a Care Pause has been commanded via a single Care Pause button and is in effect for both the bed exit and the bed status monitoring, in an example embodiment;

FIG. 24 is a screenshot of a status board for a monitoring application in an example embodiment; and

FIG. 25 is a screenshot of a detail page for one bed of the status board of FIG. 24 in an example embodiment.

It will be noted that throughout the appended drawings, like features are identified by like reference numerals. To not unduly encumber the figures, some elements may not be indicated in some Figures if they were already identified in a preceding figure. It should be understood herein that elements of the drawings are not necessarily depicted to scale. Some mechanical or other physical components may also be omitted in order to not encumber the figures.

### DETAILED DESCRIPTION

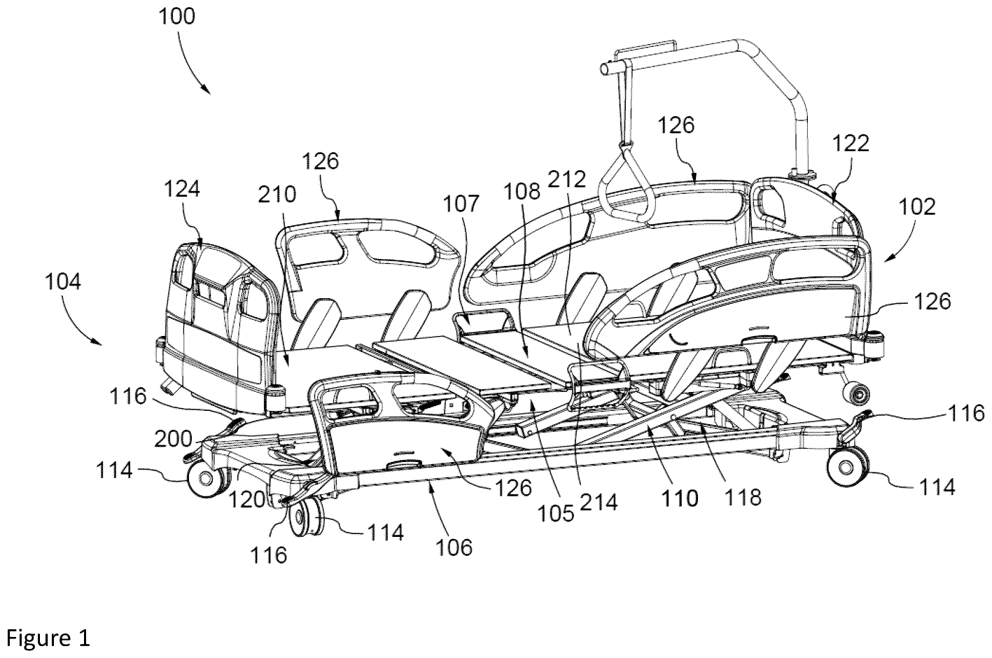

A patient support apparatus 100 in accordance with an embodiment of the present technology is illustrated in FIGS. 1 to 5. The patient support apparatus 100 may be used in a medical setting for supporting a patient. In this embodiment, the patient support apparatus 100 is a hospital bed 100 that is used in a hospital, and in particular in an intensive care unit (ICU) or medical-surgical (med-surg) setting. It is contemplated that, in other embodiments, the patient support apparatus 100 may be a different type of patient support apparatus such as, for example, a stretcher, a motorized chair, an operating room table, or other specialty tables (e.g., an examination table).

With reference to FIG. 1, the bed 100 has a head end 102 and a foot end 104 opposite each other and defining a length of the bed 100 therebetween. As will be appreciated, in use, when the patient is lying on the bed 100, the patient's head is closer to the head end 102 while the patient's feet are closer to the foot end 104. The bed 100 also has left and right sides 105, 107 extending between the head end 102 and the foot end 104 and defining a width of the bed 100 therebetween.

Some of the structural components of the bed 100 will be designated hereinafter as “right”, “left”, “head” and “foot” from the reference point of an individual lying on their back on the bed 100 with their head oriented toward the head end 102 of the bed 100 and their feet oriented toward the foot end 104 of the bed 100. Similarly, the term “headward” refers to an element located towards the head end 102 of the bed 100 and the term “footward” refers to an element located towards the foot end 104 of the bed 100. Furthermore “interior” and “exterior” views are also designated from the reference point of the patient lying in the bed. Therefore, an interior view shows an element as seen by the patient looking toward the environment outside of the bed and an exterior view shows an element as seen by a person outside of the bed. Generally, an exterior view shows the exterior surfaces of the bed, and the interior view shows the interior surfaces of the bed. The terms “inner” and “outer” will similarly be used to describe the position of elements relative to the bed.

The bed 100 has a base 106 and a patient support assembly 108 operatively connected to the base 106. Four casters 114 are connected to the base 106 at respective corners thereof to allow the bed 100 to be moved along a floor. Additional casters may be provided in other embodiments. Respective brakes 116 are provided for each caster 114 to selectively lock and unlock the casters 114. The bed 100 may also have a drive wheel (not shown) connected to the base 106 for driving the bed 100 on the floor.

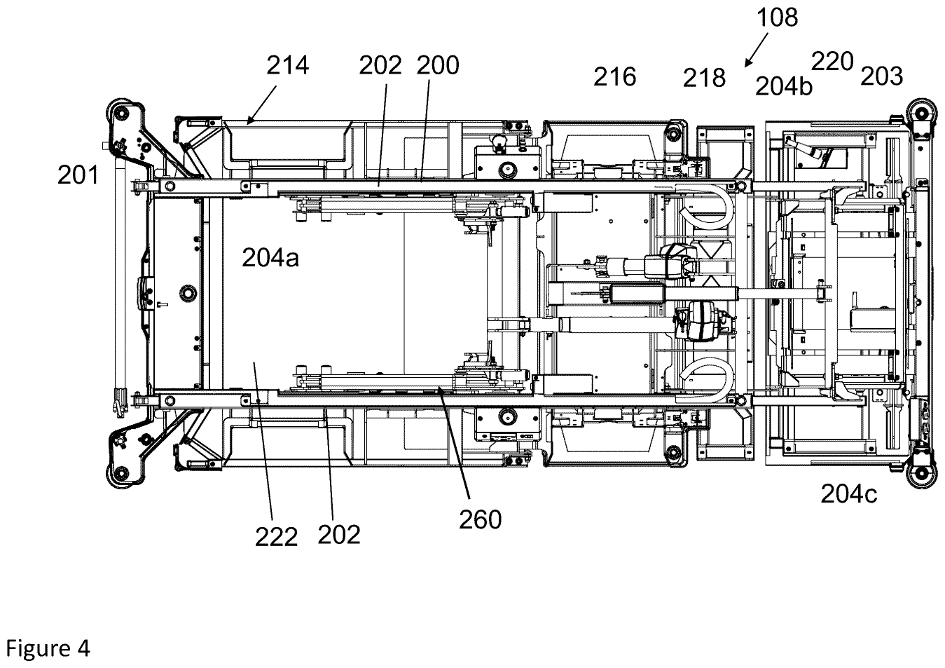

The patient support assembly 108, which is disposed above the base 106, is configured to accommodate the patient thereon and includes an upper frame 200 and a deck 210 supported by the upper frame 200. The upper frame 200 extends from a head end 201 to an opposite foot end 203. As shown in FIG. 4, in this embodiment, the upper frame 200 has left and right longitudinal rails 202 that extend parallel to each other and are interconnected by a plurality of transversal members 204a, 204b, 204c extending therebetween. The upper frame 200 may be configured in any suitable manner in other embodiments.

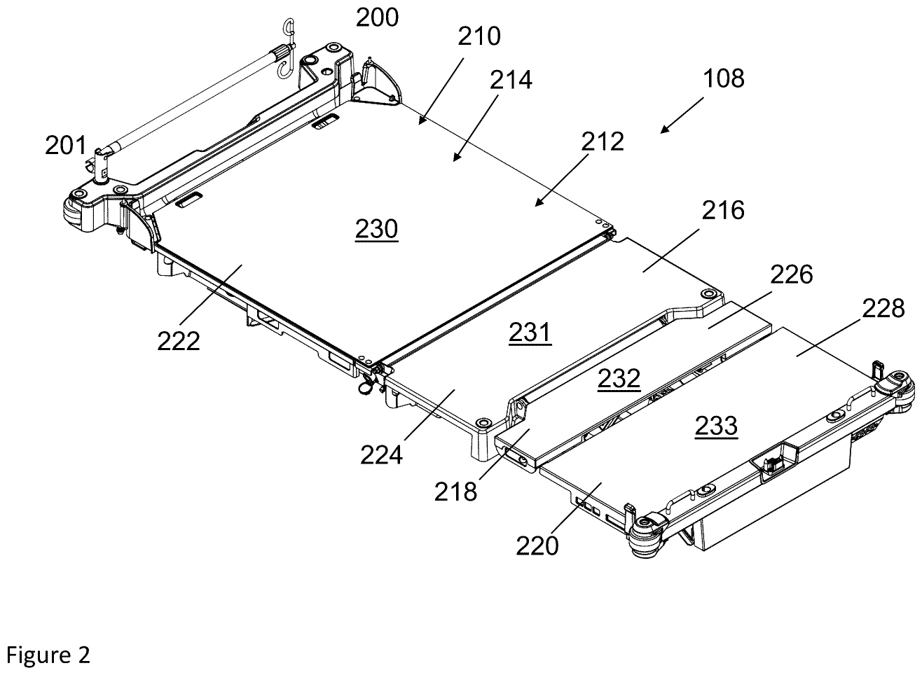

With reference to FIG. 2, deck 210 defines a top face 212 on which a support surface such as a mattress or the like is supported. Deck 210 includes a plurality of deck sections that are connected to the frame 200 and, in use, support different areas of the patient's body. Notably, the deck sections forming the deck 210 include an upper body deck section 214 for supporting an upper body of the patient, a seat deck section 216 for supporting the patient's upper legs area, a thigh deck section 218 for supporting the patient's thighs, and a foot deck section 220 for supporting the patient's lower legs and feet. The deck sections 214, 216, 218, 220 are longitudinally consecutive to each other and together form the top face 212. Notably, the upper body deck section 214, seat deck section 216, thigh deck section 218 and foot deck section 220 have an upper body deck section panel 222, a seat deck section panel 224, a thigh deck section panel 226 and a foot deck section panel 228, respectively. The deck section panels 222, 224, 226, 228 define respective top faces 230, 231, 232, 233 such that, together, they form the top face 212 of the deck 210.

The deck sections 214, 216, 218, 220 can be articulated in such a manner as to optimize comfort for the patient lying on the bed 100. The deck sections 214, 216, 218, 220 are movably connected to each other. In particular, the upper body deck section 214 and the thigh deck section 218 are pivotably connected to the seat deck section 216. The foot deck section 220 is pivotably connected to the thigh deck section 218. The seat deck section 216 is fixed to the upper frame 200 but is also movable with a movable portion of the upper frame 200. In this embodiment, the movement of the foot deck section 220 as the bed 100 transitions between different positions is dependent on the movement of the other deck sections, namely of the thigh deck section 218 to which it is connected. That is, in this embodiment, the foot deck section 220 is not directly acted upon by an actuator external to the foot deck section 220 in order to change its position. Instead, the foot deck section 220 follows the motion described by the actuation of the thigh deck section 218. Example embodiments for the articulation of deck sections are known in the art and some are described in greater detail in International Patent Application Publication No. WO2024154074, by the same Applicant, the entirety of which is incorporated by reference herein. It is contemplated that fewer deck sections may be provided in other embodiments (e.g., three deck sections).

Referring back to FIG. 1, the bed 100 has a plurality of barriers generally disposed around the patient support assembly 108 including endboards, namely a headboard 122 at the head end 102 and a footboard 124 at the foot end 104, and at least one siderail 126 on the left and right sides 105, 107. The siderails 126 can be raised to prevent the patient from falling off the bed 100 or lowered (as shown for one of the siderails 126 in FIG. 1) to allow the patient to exit the bed 100 and/or to allow care providers to access and attend to the patient. In the embodiment shown in FIG. 1, the siderails 126 include two siderails on each side (a head siderail and a foot siderail).

An elevation system 110 operatively connects the patient support assembly 108 to the base 106 to move the patient support assembly 108 relative to the base 106, namely selectively raising or lowering the patient support assembly 108 relative to the base 106. In addition to raising and lowering the patient support assembly 108, the elevation system 110 also allows the bed 100 to assume various different positions such as a flat horizontal position (shown in FIG. 1), a Trendelenburg position, a reverse Trendelenburg position, a cardiac chair position and a full chair position. It is contemplated that, in other embodiments, the bed 100 may be configured to assume more or fewer positions. For instance, in some cases, the bed 100 may permanently remain in the flat horizontal position and simply be raised and lowered by the elevation system 110.

In this embodiment, the elevation system 110 includes a head end lift assembly 118 and a foot end lift assembly 120 which connect the base 106 to a head end portion and a foot end portion of the upper frame 200, respectively. Each of the head end lift assembly 118 and the foot end lift assembly 120 may be raised to a fully extended and lowered to a fully collapsed position. The lift assemblies 118, 120 may be controlled symmetrically so that that the lift assemblies 118, 120 are in equivalent extended positions at the same time (e.g., both in the fully extended position or both in the fully collapsed position), or they may also be controlled asymmetrically so that they are in different extended positions at the same time. For example, in some cases, the head end lift assembly 118 may be in the fully extended position while the foot end lift assembly 120 is in the fully collapsed position or vice-versa (e.g., in the Trendelenburg position).

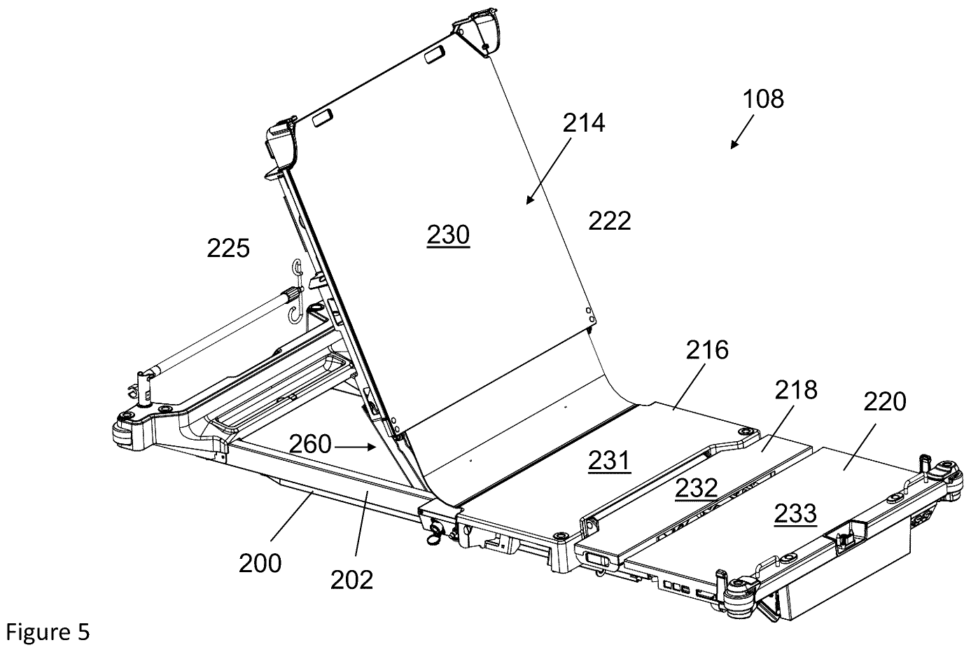

With reference to FIG. 5, the upper body deck section 214 has a supporting structure 225 and an articulation mechanism 260 that operatively connects the supporting structure 225 to the upper frame 200 of the bed 100 to allow motion of the supporting structure 225 relative thereto. More particularly, the articulation mechanism 260 enables the upper body deck section 214, including the supporting structure 225, to move relative to the upper frame 200 between a lowered position (FIGS. 1 to 4) and a raised position (FIG. 5), and passing through various intermediate positions therebetween. In the illustrated embodiment, the lowered position is a horizontally flat position. In the illustrated embodiment, the raised position shown in FIG. 5 is considered to be a fully raised position. It will be readily understood that other embodiments could provide a lowered and a fully raised position at a different angle with respect to the horizontal than what is shown in these figures. Other articulation mechanisms are provided for at least some of the other deck sections.

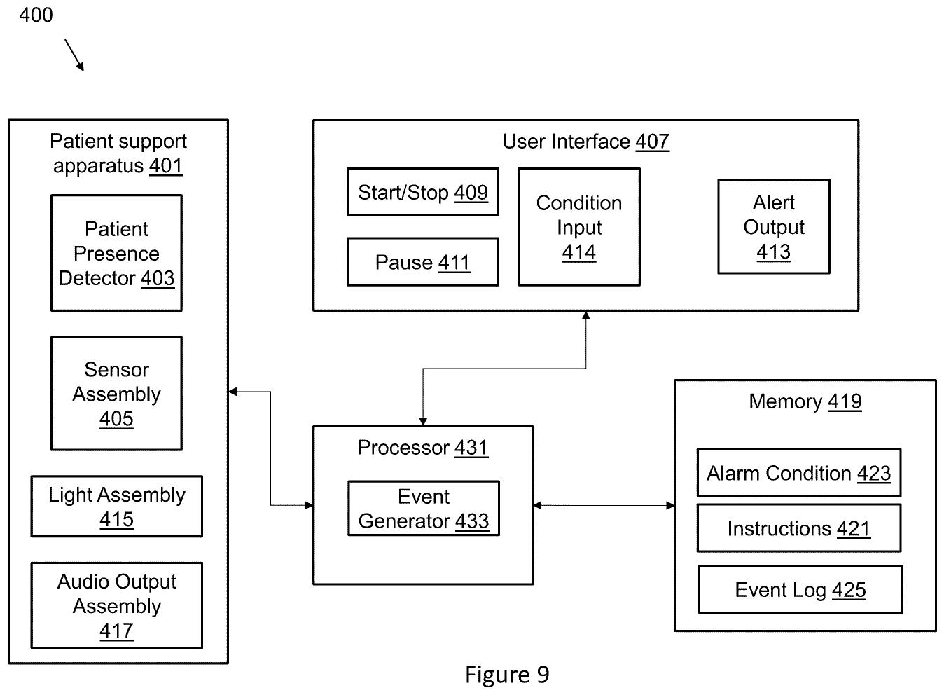

The base 106 further comprises sensors for determining that a weight is present on the bed. These sensors may be used by the patient presence detector 403 shown in FIG. 9. In one example embodiment, a plurality of load cells are adapted to provide an indication of the weight on the bed 100. The one or more load cells are operatively connected to a controller. The patient presence detector 403 may also be used for determining a position of the patient in the bed 100, a center of mass of the weight present on the bed, a level of activity of the patient and a load distribution of the weight of the patient.

In one or more embodiments, the bed 100 includes four load cells, where each load cell is positioned at a respective corner i.e., foot right (FR), foot left (FL), head right (HR) and head left (HL). Each load cell is operable to generate a signal comprising a voltage value upon presence of a weight on the bed, which is converted into a respective weight value for each load cell received by the controller. The respective weight values may be used to determine the weight of the person or the object(s) on the bed 100, a position and/or movement of the center of gravity on the bed 100, and/or further processed to determine patient agitation and activity, vital signs (e.g., heart rate (HR) and respiratory rate (BR)) and the like. In one or more alternative embodiments, the bed 100 may include a different number of load cells, such as a single load cell.

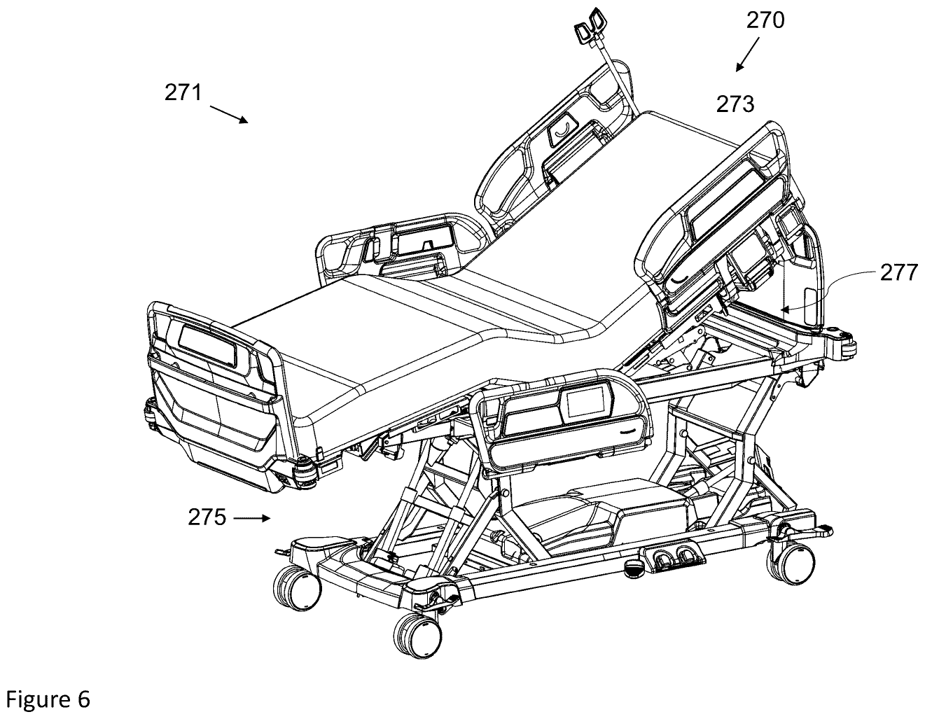

FIG. 6 is a perspective view of patient support apparatus 270 in accordance with another embodiment. The bed 271 is shown equipped with a patient support surface, namely mattress 273. The mattress 273 is typically removably attached to the hospital bed. The patient support assembly 277 and elevation system 275 are shown in a chair configuration with the deck sections angled appropriately.

The bed has many other features that will not be described in detail herein as they are well known in the art. The above description of the bed is thus not meant to be exhaustive but rather to provide sufficient context for the reader.

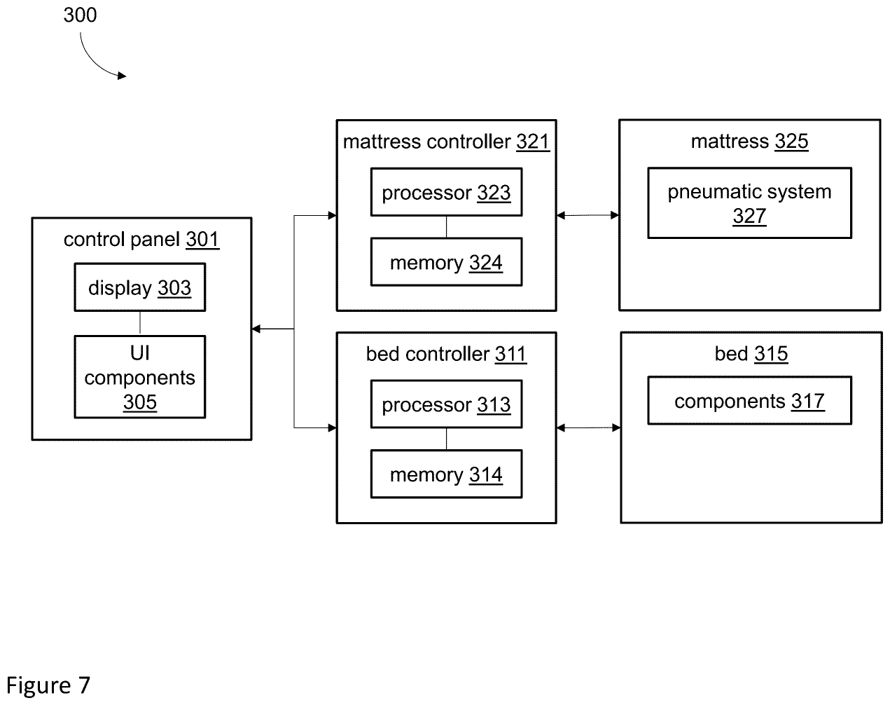

With reference to FIG. 7, there is shown a schematic diagram of main control components 300 of a bed 315 and inflatable mattress 325 (also shown schematically). A control panel 301 is operatively connected to a bed controller 311 and mattress controller 321 in accordance with one or more non-limiting embodiments of the present disclosure.

The control panel 301, also referred to as control interface, user interface, and input device, enables a user (e.g., caregiver) to interact with the different components 317 of the hospital bed 315 and/or mattress 325 by displaying information and transmitting instructions to control different components and functions of the hospital bed 315 and/or mattress 325.

The control panel 301 may be mounted on or may be adjacent to the hospital bed 315. It will be appreciated that one or more control panels with different components may be mounted on siderails, endboards (headboard, footboard) and/or fixed or removable panels of the hospital bed 315. It will be appreciated that the control panel 301 may be provided at different locations, may be integrated into the or may be a separate device operatively connected to at least one component of the bed.

### Control Panel

The control panel 301 includes various user-interface (UI) components such as, but not limited to, one or more of: visual components (e.g., displays and lights), audio components (e.g., speakers and microphones), control or input components (e.g., buttons, knobs, switches, keypads, feedback components and touchscreens), sensors (e.g., proximity sensors) and additional input/output interfaces and communication components (e.g., connectivity and charging ports, wireless connectivity modules). As shown schematically in FIG. 7, the control panel 301 includes a display 303 and UI components 305.

The one or more displays (shown as display 303 in FIG. 7), also referred to as display screen(s), display device(s), display unit(s) or display interface(s), may include one or more of a liquid crystal display (LCD), light emitting diode (LED) display, organic light emitting diode (OLED) display, plasma displays, e-ink (electronic Ink) display, touch screen displays, quantum dot displays, digital light processing (DLP) projector, head-up displays (HUD), virtual reality (VR) headset displays, and augmented reality (AR) displays.

In one or more embodiments, the control panel 301 is configured to display graphical user interfaces (GUIs) to control and/or view different data relating to the hospital bed 315, the mattress 325 and/or the patient on the display 303.

A given user may interact with the GUIs displayed on the control panel 301 by using the display 303 (e.g., if implemented as a touchscreen), UI control components (e.g. button, knobs, switches), voice, or gestures.

The control panel 301 is configured for receiving data signals from and transmitting signals to the bed controller 311 and the mattress controller 321.

The received data may include for example feedback and error notifications. The feedback is transmitted from the controllers 311, 321 over the communication pathway and processed by the control panel 301 to update its display 303 or alert the user.

### Controllers

The control panel 301 is operatively connected to the bed controller 311 and the mattress controller 321. In some embodiments, the bed controller 311 and the mattress controller 321 may be implemented as a single controller. In one or more other embodiments, the control panel 301 may be connected to other types of controllers.

The bed controller 311 is operatively connected to various systems, subsystems and components of the hospital bed 315, which may include, as a non-limiting example, adjustment mechanisms, actuators, sensors, integrated weighing systems, alert indicators, and the like. In one or more embodiments, the bed controller 311 may be integrated or attached to the hospital bed 315.

The bed controller 311 includes at least a processor 313 and a memory 314. Additionally, the mattress controller 321 may include or be operatively connected to one or more input/output interfaces and communication interfaces (not shown).

The mattress controller 321 is operatively connected to components of the pneumatic system 327 of the mattress, which may include, as a non-limiting example, blowers, pressure sensors, valves, blowers, sensors and the like. The mattress controller 321 is configured to receive inputs (e.g., from the control panel 301 or the bed 315), process the inputs and provide outputs (e.g., to the control panel 301, to the hospital bed, or pneumatic system components). In one or more embodiments, the mattress controller 321 may be located within mattress 325. In other embodiments, the mattress controller 321 may be located outside of the mattress, such as within or mounted to the hospital bed 315.

The mattress controller 321 includes at least a processor 323 and a memory 324. Additionally, the mattress controller 321 may include or be operatively connected to one or more input/output interfaces and communication interfaces (not shown).

It should be understood that the bed controller 311 and mattress controller 321 may have different types and/or number of components.

In one or more implementations, the processors 313, 323 which may also be referred to as processing devices, and processing units, may each include a single-core microprocessor. In one or more other implementations, a given one of the processors 313, 323 may include a multi-core microprocessor. In one or more alternative implementations, a given one of the processors 313, 323 may include one or more of: a microcontroller, a digital signal processor (DSP), an integrated circuit purposed for specific operations within an embedded system, a system on a chip (SoC), a field-programmable gate array (FPGA), and an application-specific integrated circuit (ASIC) configured to carry out the processing and functionalities described herein.

The memories 314, 324 may include volatile and non-volatile memories. In one or more implementations, a given one of the memories 314, 324 may include volatile memory, such as random-access memory (RAM), and/or alternatively static random-access memory (SRAM) or dynamic random-access memory (DRAM). In one or more implementations, a given one of the memories 314, 324 may include non-volatile memory, such as flash memory and/or alternatively electrically erasable programmable read-only memory (EEPROM) or ferroelectric RAM (FRAM). The memories 314, 324 are configured to store computer-readable instructions executable by the processors 313, 323 to carry out the processing and functionalities described herein.

The one or more communication interfaces may include wired and wireless communication interfaces to connect the components of the hospital bed 315 to other medical devices, computing devices (e.g., wireless modules, nurse station computer, server(s) and mobile devices), and communication networks (e.g., hospital network or cellular network) to transmit an/or receive data.

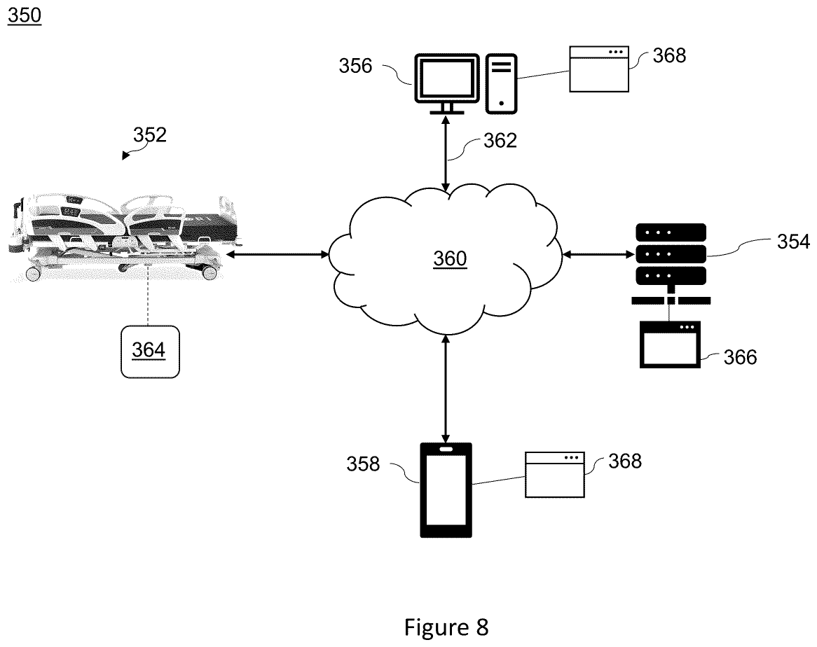

With reference to FIG. 8, there is shown an environment and system 350 comprising a patient support apparatus 352, a server 354, a nurse station computer 356, and a client device 358 coupled to one or more communication networks 360 via respective communication links 362 (not separately numbered).

The patient support apparatus 352 may be located in a healthcare facility. The patient support apparatus 352 may be for example located within a zone of a room, a hallway, an intensive care unit, an emergency room, an operating room, and the like. The patient support apparatus 352 or other form of patient support apparatus could be used in various locations without departing from the scope of the present technology.

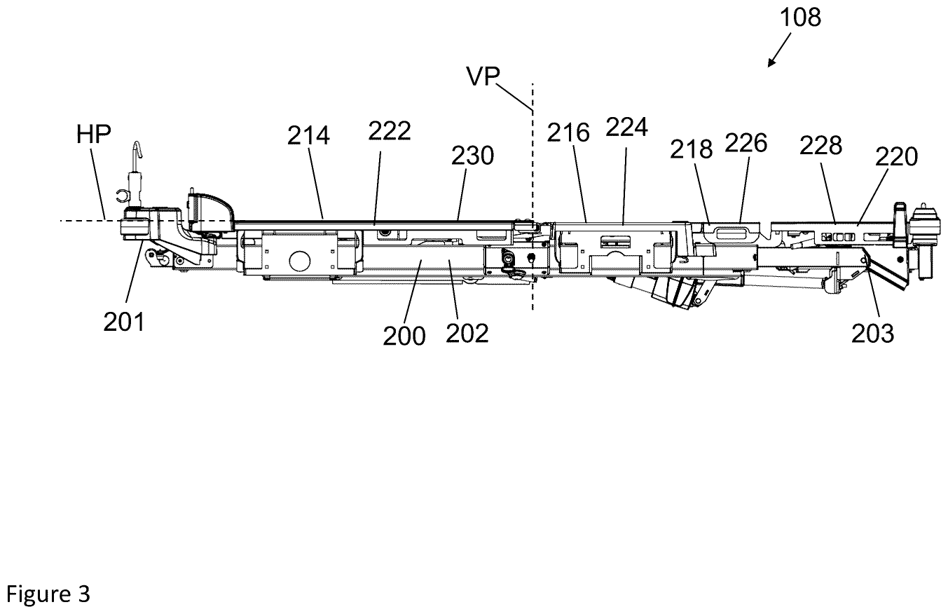

The patient support apparatus 352 comprises a controller 364. The controller 364 may be similar to bed controller 311 of FIG. 3 and include at least a portion of the components of a computing device, such as one or more processing units, one or more memories, and one or more communication interfaces.

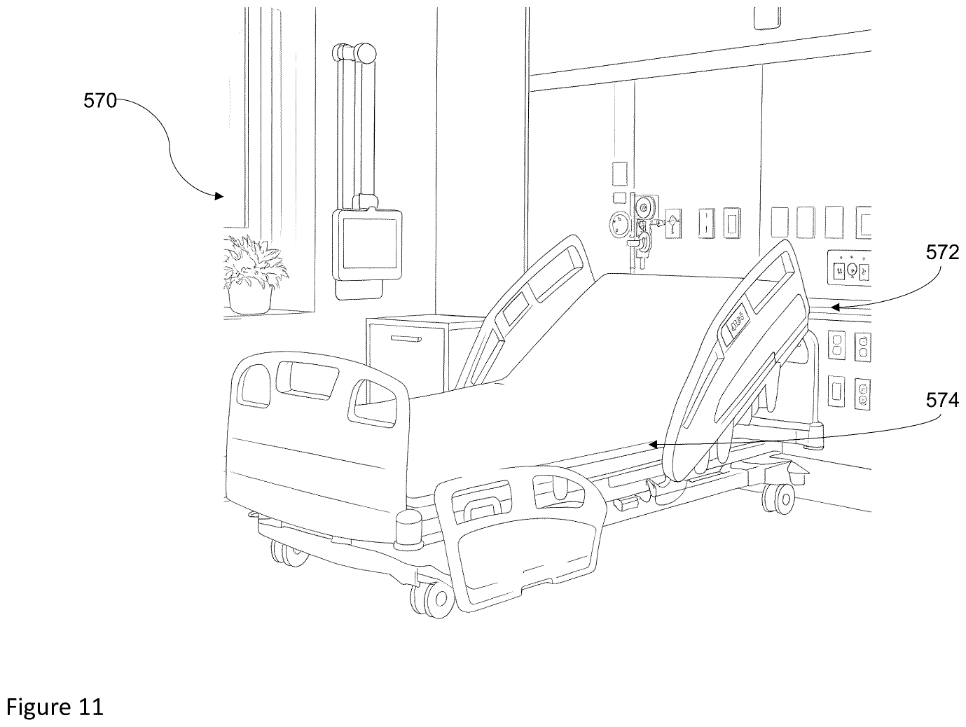

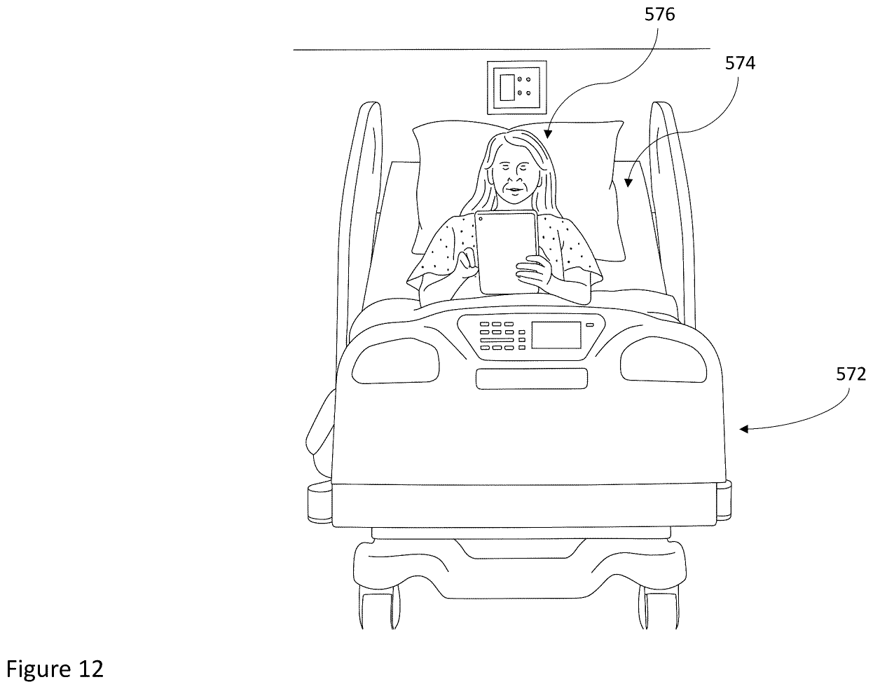

In some implementations, the patient support apparatus 352 may communicate with a headwall (see FIGS. 11 and 12) located in a room. The headwall and patient support apparatus 352 may be configured to transmit and/or receive data, for example nurse call communications. Additionally, or alternatively, the patient support apparatus 352 may connect to a hospital network, a nurse call interface, and other devices via a wired or wireless communication link.

The server 354 is configured to inter alia: (i) receive data from and transmit data to the patient support apparatus 352; (ii) receive data from and transmit data to the nurse station computer 356; (iii) receive data from and transmit data to the client device 358; (iii) execute the monitoring functionality 366.

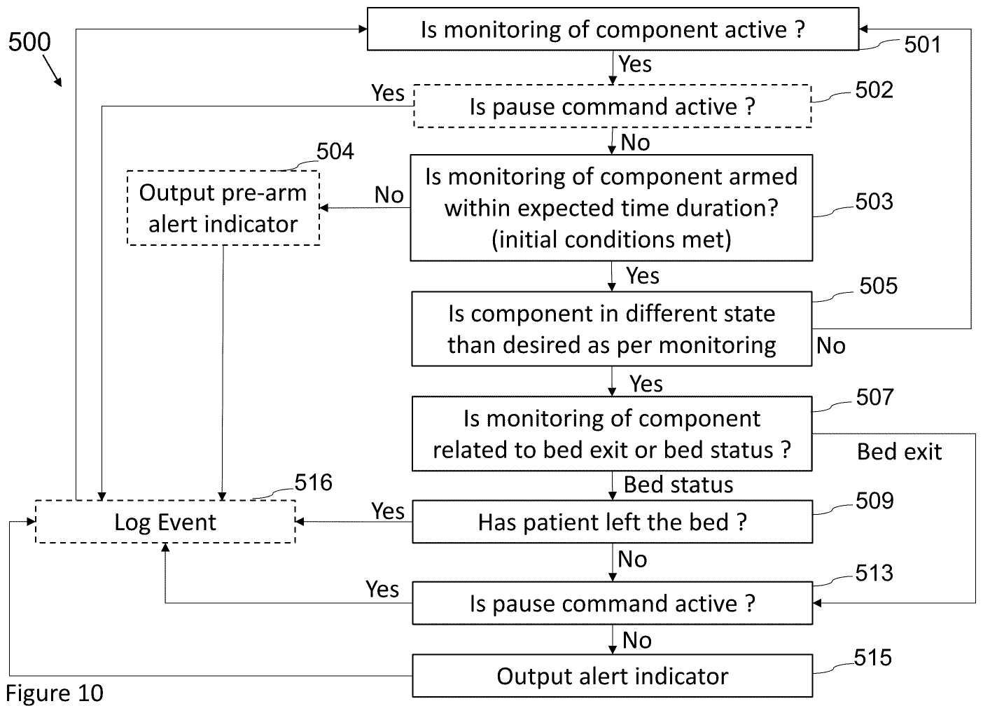

The server 354 executes the monitoring functionality 366. As part of the monitoring functionality 366, the server 354 is configured to implement a monitoring system as will be described with reference to FIGS. 9 and 10.

In one or more embodiments, the server 354 is configured to provide a graphical user interface (GUI) via the monitoring functionality 366, which enables to receive and display data related to the subsystems and components of the patient support apparatus 352 (e.g., bed status, bed exit, alerts, etc.) and transmit control data to at least some of the subsystems and components of the patient support apparatus 352 via the GUI.

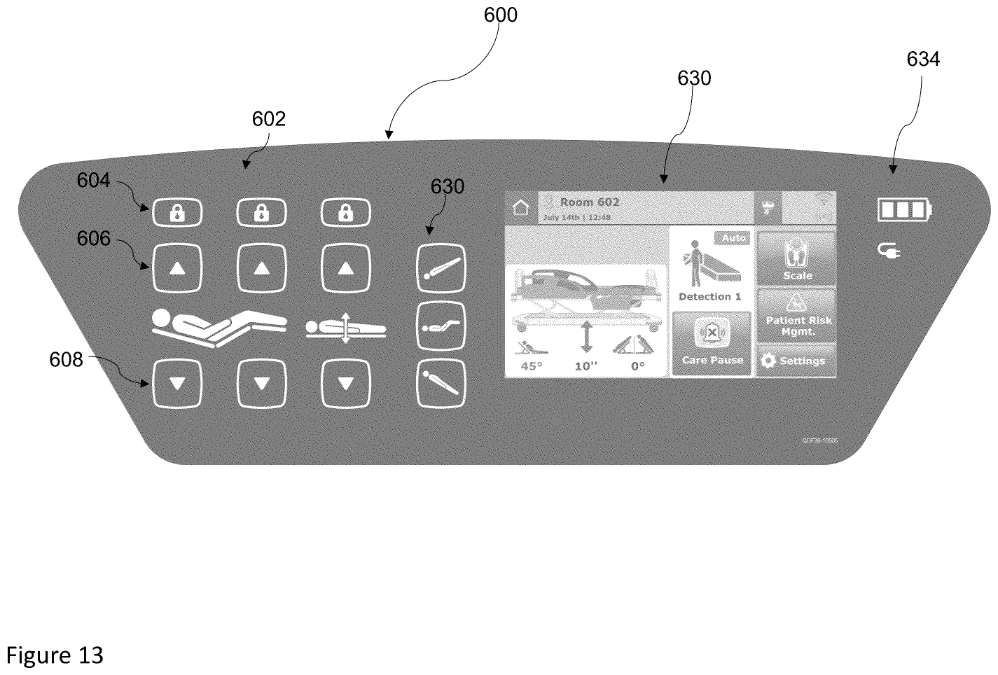

The server 354 is configured to process numerical data and transform the numerical data into multiple forms of visual representations. The visual representations may include, but are not limited to, line plots, bar graphs, pie charts, scatter plots, heat maps, histograms, and color-coded diagrams. Additionally, the server 354 is configured to generate visualizations like 2D and 3D graphs, time lapses, interactive plots, and real-time data visualizations. The generated visual data is then displayed as a part of the GUI, which can be accessed by users through computing devices and/or display units such as the nurse station computer 356, and the client device 358, for example via monitoring application 368. The monitoring application 368 enables users to interact with the visual data, as a non-limiting example via functionalities like zooming, panning, and selecting specific data points for a more detailed view, or program functionalities of the patient support apparatus 352. Non-limiting examples of GUIs generated by the server 354 are shown in FIGS. 13 to 25.

The server 354 is configured to transmit notifications via the one or more communication network 360. In one or more embodiments, the server 354 is configured to transmit notifications to the monitoring application 368 of the client device 358 and of the nurse station computer 356 and to the patient support apparatus 352.

In some implementations of the present technology, the server 354 may be connected to the communication network 360 via a communication link 362. In alternative implementations of the present technology, the server 354 may be optional.

The implementation of the server 354 is well known to the person skilled in the art of the present technology. The server 354 may be implemented as one or more computing devices, e.g., one or more processors (e.g., central processing unit (CPU) and/or graphics processing unit (GPU)), a memory and/or storage unit, input/output interfaces and communication interfaces. In one or more implementations, the server 354 may be implemented as part of a cloud system (not shown).

It will be appreciated that the server 354 may provide the output of one or more processing steps to another computing device for display, confirmation and/or troubleshooting. As a non-limiting example, the server 354 may transmit data including calculated values, results, and machine learning parameters, for processing and/or display on a computing device such as a smart phone, tablet, and the like.

The nurse station computer 356 is a centralized computing system configured to manage and access patient information, coordinate care, and to enable and facilitate communication among healthcare professionals in a healthcare facility. The nurse station computer 356 is configured to store electronic medical records (EMRs), scheduling and tracking patient appointments, integrating with hospital-wide communication and monitoring systems, assisting in medication management, and providing tools for reporting and analytics.

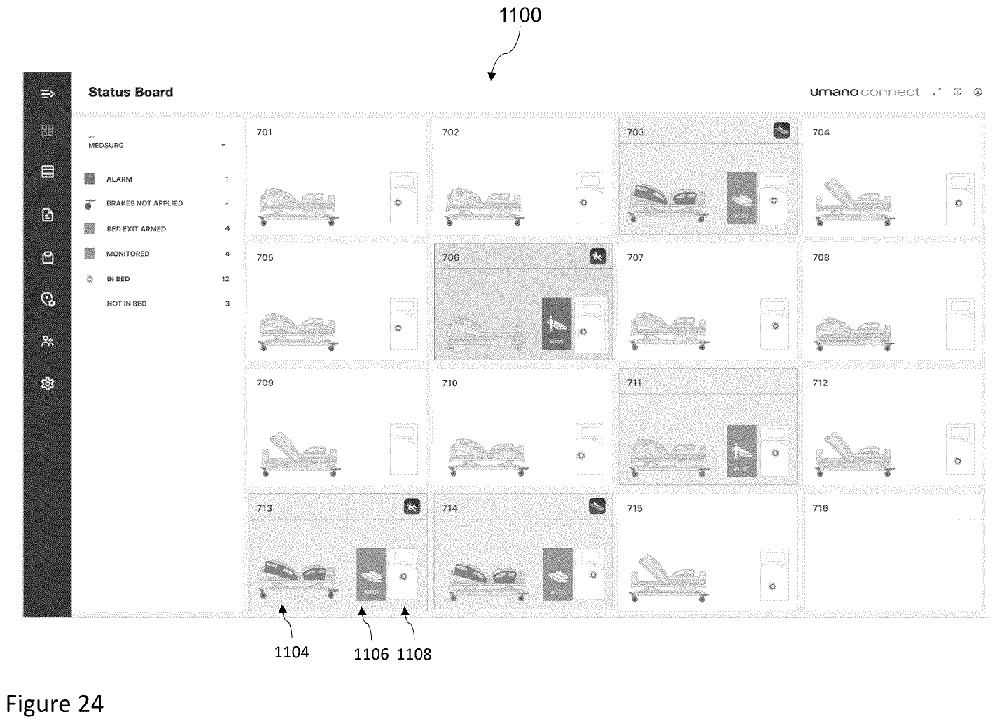

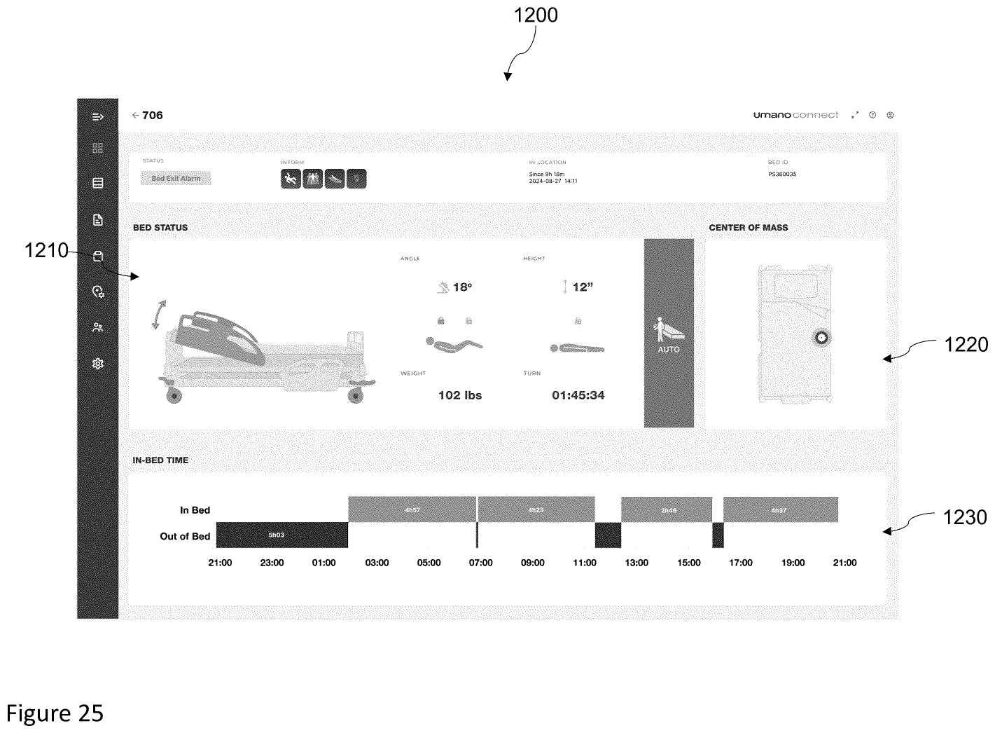

In one or more implementations of the present technology, the nurse station computer 356 is configured to execute the monitoring application 368. The monitoring application 368 may be executed as a stand-alone software application, as an application or may be accessible via a browser application (not shown). Non-limiting examples of interfaces of the monitoring application 368 are shown in FIGS. 24 and 25.

In one or more embodiments, the nurse station computer 356 is configured to: (i) receive data from the patient support apparatus 352 and/or the server 354; and (ii) transmit data to the patient support apparatus 352 and/or the server 354.

As a non-limiting example, the nurse station computer 356 may receive and display, via monitoring application 368, data related to the subsystems and components of the patient support apparatus 352 (e.g., bed status, bed exit, alerts, etc.) and transmit control data to at least some of the subsystems and components of the patient support apparatus 352.

The environment and system 350 comprise one or more client devices 358 (only one shown in FIG. 8).

The client device 358 is associated with one or more users (not shown), such as medical personnel or caregivers. As such, the client device 358 can sometimes be referred to as a “computing device”, “end user device” or “client computing device”.

As shown in FIG. 8, the client device 358 may be a tablet used by one or more users, such as medical staff. It will be appreciated that the client device 358 may be implemented as a server, a desktop computer, a laptop, a smartphone, a tablet, and the like without departing from the scope of the present technology.

The client device 358 is configured to execute monitoring application 368.

In one or more implementations, the client device 358 is configured to access monitoring application 368 via a browser application (not shown). How the given browser application is implemented is not particularly limited. Non-limiting examples of the given browser application that is executable by the client device 358 include GOOGLE Chrome™ MOZILLA Firefox™, MICROSOFT Edge™, and APPLE Safari™.

In one or more embodiments, the client device 358 is configured to: (i) receive data from the patient support apparatus 352 and/or the server 354; and (ii) transmit data to the patient support apparatus 352 and/or the server 354.

As a non-limiting example, the nurse station computer 356 may receive and display, via monitoring application 368, data related to the subsystems and components of the patient support apparatus 352 (e.g., bed status, bed exit, alerts, etc.) and transmit control data to at least some of the subsystems and components of the patient support apparatus 352.

Additionally, the environment and system 350 may comprise one or more medical devices and/or computing devices (e.g., mobile device such as a phone, tablet, etc.) connected to the one or more communication networks 360 or directly to components of the environment and system 350.

The one or more communication networks 360 may include one or more of wireless local area networks (WLANs), wired local area networks (LANs), personal area networks (PANs), nurse call systems, wide area networks (WANs), cellular networks, internet of things (IoT) networks, mesh networks, virtual private networks (VPNs), optical fiber networks, and digital health platforms.

How a given communication link 362 (not separately numbered) between the patient support apparatus 352, the server 354, the nurse station computer 356, and the client device 358 is implemented will depend inter alia on how each computing device is implemented. It will be appreciated that each given communication link 362 may be of a different type (e.g., wired or wireless) and may form or connected to a different type of network of the one or more communication networks 360.

A monitoring system 400 for a patient support apparatus 401 having a patient receiving surface in accordance with one or more embodiments will now be described with reference to FIG. 9.

The monitoring system 400 has a patient presence detector 403 operatively connected to the patient support apparatus 401 to detect a presence of a patient on the patient receiving surface. The patient presence detector 403 may be implemented as a combination of hardware and software components. In an example embodiment, the patient presence detector 403 determines if a patient is present on the patient receiving surface using prior art techniques. While the patient presence detector 403 awaits to detect the presence of a patient on the bed, the bed can be considered to be in “standby” state. The patient presence detector 403 is generally equipped with one or more sensors to detect presence of the patient. For example, the sensors may include load cells.

If the presence of a patient is detected using the detection of a presence of a weight on the bed, one particular technique may involve determining that a weight larger than a predetermined threshold weight is present on the bed. For example, the patient presence detector 403 may determine that a patient is present on the bed if the detected weight is above 22 kg. In another example embodiment, the patient presence detector 403 may determine that a patient is present on the bed if the weight distribution detected on the patient receiving surface covers an area greater than a threshold area using prior art techniques. In such embodiments, the patient presence detector 403 may include one of: deformation sensors, strain gauges and load cells.

The presence of a patient on the bed may be determined by other means. In one embodiment, the patient presence detector 403 may include a temperature sensing assembly. A temperature sensor may be provided on or near the patient support apparatus, on or near the patient receiving surface or further away from the patient such as affixed to a wall or ceiling. In another embodiment, a camera or a thermal camera may be used to detect the presence of the patient. In still another embodiment, a vital signs system may be used to validate that the weight or mass detected on the patient support surface is a living being and not a heavy piece of equipment. In alternative embodiments, presence of the patient on the bed may be detected via patient identification technologies using radio-frequency technologies by detecting a tag worn by a patient via sensors located attached to or in proximity of the bed (e.g., ultrawideband (UWB), Bluetooth, and radio-frequency identification (RFID) tag). In other embodiments, for example when the patient support surface is a mattress with pressure sensors, presence of the patient may be detected via pressure readings.

The monitoring system 400 has a sensor assembly 405 operatively connected to the patient support apparatus 401 used to determine a state of one or more components of the patient support apparatus 401. Prior art techniques are used by the sensor assembly 405 to determine the state of the component(s) of the patient support apparatus 401. The sensor assembly 405 obtains an indication for the state of the component. It will be appreciated that depending on the configuration of a given sensor in the sensor assembly 405, the determination of the status of the component may be directly provided by the sensor of the sensor assembly, or sensor measurements indicative of the state of the component are provided by the sensor assembly 405, which in turn enable the processor to determine the state of the component based on the sensor measurements (e.g., based on a numerical value). Non-limiting examples of sensors in the sensor assembly 405 may include for position/angle: limit switches, reed switches, inductive/capacitive proximity sensors, optical interrupters, Hall-effect sensors, potentiometers, optical/magnetic encoders, LVDTs, magnetostrictive linear sensors, inclinometers/IMUs, and ultrasonic/IR/ToF distance sensors; for load/force: strain-gauge load cells, bonded strain gauges, force-sensing resistors, inline torque sensors, pneumatic/hydraulic pressure sensors, flow sensors, and cable-tension sensors; motion: tachometers, accelerometers, and gyroscopes; for presence/interlocks: latch/lock switches, caster-brake sensors, tamper switches, RFID/NFC readers, and contact-edge sensors; for electrical/thermal: current sensors, voltage monitors, temperature sensors, and humidity/wetness sensors; and for imaging/depth: 2D/3D cameras. The sensor assembly 405 could be integrated into the bed and/or the mattress.

Example desired states and components include an indication of the elevation of the bed from the floor (hi-low adjustment) as a distance from the floor or as a minimum or maximum, the length of the bed and/or the mattress, the width of the bed and/or the mattress, the angles of the head, back, seat, thigh, leg and foot decks, rests or panels, the position (raised, lowered) of each siderail or of combinations of siderails (foot/head or left side/right side or all four siderails), the presence of endboards (headboard, footboard) in their sockets, the command and/or the angle of the tilt of the deck for foot or head elevation (Trendelenburg and Reverse Trendelenburg Adjustment), the command and/or the angle of the tilt of the deck for left or right side elevation (lateral tilting), the head-of-bed (HOB) angle (absolute angle from the horizontal which can be a combination of a backrest angle and a Trendelenburg or Reverse Trendelenburg position), the activation of a lock for the movement of the deck or the elevation, a change in position of the combined bed and mattress (flat horizontal position, Trendelenburg position, a reverse Trendelenburg position, a cardiac chair position and full chair position), the detected weight on the bed, the distribution of the weight on the bed (including center of gravity on the bed), a change in detected weight on the bed, the presence of the patient in a lateral or longitudinal zone of the bed, a change in the level of detection of the bed exit, a maximum duration for the in bed time, a level of activity of the patient in the bed, a change in the volume of audible alerts, a change in the configuration of messages displayed on the user interface, the inflation status of each zone of the mattress, the level of immersion of the patient on the mattress, the activation, deactivation, duration or settings of specific functions of the mattress, such as percussion/vibration, alternating pressure, turn assist, continuous lateral rotation therapy (CLRT), prone therapy, side exit assistance, max inflate, full deflate, vacuum and low air loss, the level of humidity under the patient, the presence of an IV drip pole in its socket, the presence of an oxygen cylinder in its bracket, the presence of a patient lift, the presence of a medical equipment on the bed, the connection of a nurse call interface to a nurse call of the facility, the connection of a personal computing device to the patient power source of the bed, the connection of an external computing device to the communications network of the bed, etc.

The monitoring system 400 has a user interface 407 for receiving inputs from a user and providing outputs to the user. The inputs are indicative of at least one of a start command to activate monitoring of the given component, an optional stop command to deactivate the monitoring of the component, a pause command to pause output of alert indicators for the activated monitoring of the component, and an optional condition of the state of the component 414. It should be understood that “a component” in the present context refers to at least one or one or more components.

The inputs are typically received via the user interface 407 (e.g., located on a headboard, footboard or siderails of the bed 100 or via a remote computing device). The inputs via the user interface 407 are provided as signals to the controller of the patient support apparatus 401. For example, the user interface 407 may include a start/stop monitoring button 409 and a pause button 411. In some embodiments, the functions of these buttons are combined and controlled by the user to indicate their selections. In other embodiments, the functions of these buttons are split, with each button indicating a single input. In some embodiments, these buttons are provided on a membrane or touchscreen on a siderail or headboard of the patient support apparatus 401 and/or via a remote application (app) that can be accessed on desktops or portable computing devices. As will be readily understood, multiple user interfaces can be provided to the user, and each serve to provide inputs to the monitoring system 400.

In some embodiments, inputs for a plurality of beds can be provided from a monitoring application (shown in FIGS. 24 and 25). For example, in a medical facility having a plurality of units and floors, administrators may determine the baseline bed status monitoring that they wish to impose depending on the types of patients that they care for. In that case, the identification of the desired state for component(s) of the bed may be carried out centrally and applied from the monitoring application to a plurality of selected beds. The inputs for the states and the components and the activation command would therefore come from the monitoring application and be communicated individually via the communications network to each selected patient support apparatus. In some embodiments, the caregivers could be given the liberty of activating monitoring for additional states and/or components but could not deactivate monitoring for the baseline components.

The outputs of the user interface 407 may be representative of an alert indicator and are typically issued by alert output 413. Alert output 413 can issue a message or a notification to be displayed on a screen provided on the patient support apparatus 401 or on a monitoring application accessible by a fixed computer or a portable device. Alert output 413 can also issue a visual and/or audible alert indicator. If the alert indicator is visual, the alert output 413 may be in communication with a light assembly 415 on the patient support apparatus for illumination of lights depending on the alert. For example, bumper lights may be available on the patient support apparatus 401 and may be illuminated in different colors, patterns or frequencies to indicate the type of alert. If the alert indicator is audible, the alert output 413 may be in communication with an audio output assembly 417 such as a loudspeaker for emission of sounds depending on the alert. The audio output assembly 417 may be provided on the patient support apparatus 401 or may be provided near the user interface 407 used for inputting commands to the monitoring system 400.

Alert indicators may only be outputted to an application and may only be visual indications provided on the application. In this embodiment, no audible alerts are indicated at or near the patient support apparatus 401 or at remote locations through the application.

The monitoring system 400 has a memory 419 for storing computer-readable instructions 421 which may be executed for enabling the various functionalities of the monitoring system 400 described herein. Memory 419 may include storage for instructions indicative of an alarm condition 423 for the state of the given component. It also may include storage for logging events monitored by the monitoring system 400 in the event log 425. It will be appreciated that memory 419 may be or may be part of memory 314 and/or memory 324 of FIG. 7.

The monitoring system 400 has a processing unit 431 operatively connected to the patient presence detector 403, the sensor assembly 405, the user interface 407 and the memory 419. The processing unit 431 is configured to determine, by executing an event detector function 433, if the alarm condition has occurred based on the state of the component and the alarm condition, and to provide an alert output to the user if the alarm condition has occurred in the presence of the patient on the patient receiving surface, in the presence of the start command from the user and in the absence of the pause command from the user. It will be appreciated that processing unit 431 may be part of processor 313 or part of processor 323.

In use, a patient 576 is assigned to their patient support apparatus 572 as shown in FIG. 12. A user, typically a caregiver mandated to care for the patient, is responsible for setting up and using a monitoring system 400 provided in association with the patient support apparatus 572. Depending on the patient's condition, one or many components of the patient support apparatus 572 are to be kept in specific states of operation. To aid the caregiver, the monitoring system 400 allows to set and activate monitoring of these components in their specific states and to issue alerts when the state of the component enters into a condition different from the desired specific state of operation. This is considered an alarm condition. As is often the case, multiple components are monitored for a single patient, with varying possible consequences on the health and safety of the patient. This can lead to multiple or repeated alerts and alarm fatigue for the caregiver. In some embodiments, monitoring conditions corresponding to sets of components may be provided as “presets” and selected by a caregiver.

The method carried out within the monitoring system 400 will now be described in more detail. A method 500 according to one embodiment will now be described in relation with FIGS. 9-10. In one or more embodiments, the steps of method 500 are executed by processor 431 using instructions 421 stored in memory 419. It will be appreciated that steps of the method 500 may also be executed by a plurality of processors.

Prior to the monitoring method 500, the caregiver will decide whether to start monitoring one or more components of the bed. If they so wish, they will use a user interface to identify the component to monitor and the desired state for the component. Only states of the components for which a measure or a state thereof can be obtained via the sensor assembly 405 can be monitored. Example states can represent a status, a position, a distance, an angle, a displacement, a speed, a locking status, a motor activation state, an actuator stroke position, a power supply state, a control input state, an occupancy/presence, an exit alert status, a surface configuration, a communication link status, a power supply status, etc.

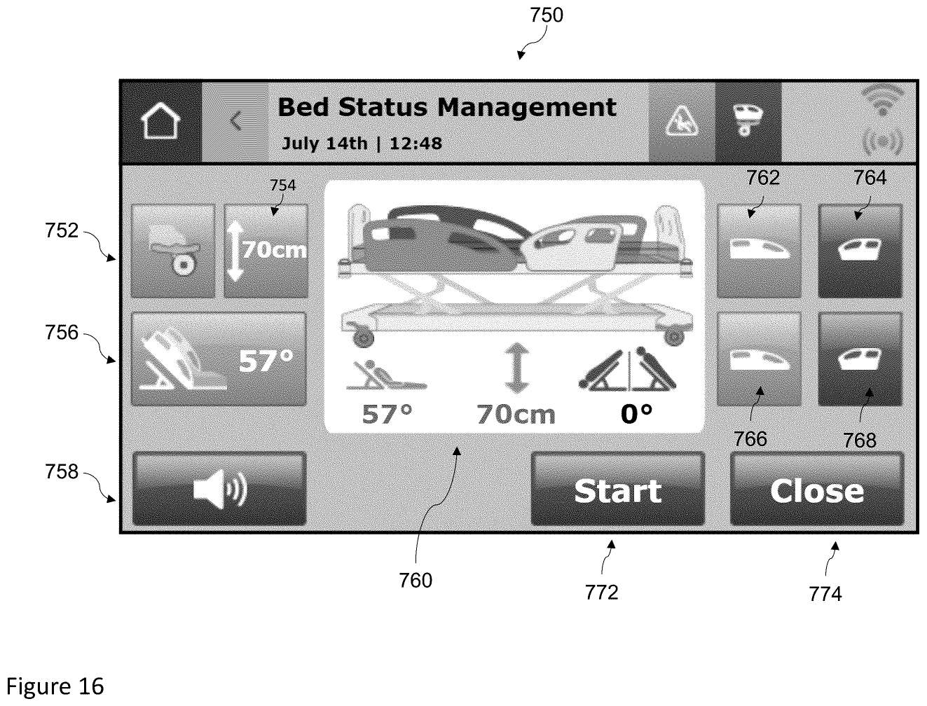

For example, if the position of a specific siderail is to be monitored, the user provides an indication of which siderail (e.g., left head siderail, left foot siderail, right head siderail end or right foot siderail) to monitor and in which position (e.g., raised, intermediate or lowered) it should be in the desired state. This can be achieved, in an example embodiment, via a touchscreen with an interactive graphical representation of the hospital bed where touching an area of the screen where a specific siderail is shown changes its display color and touching it again alternates between a raised and a lowered representation of the siderail on the bed, as shown in FIG. 16. Once the desired state of the siderail to be monitored is graphically represented, a start button is pressed to indicate that the monitoring in the represented state is active. In another example embodiment, touching a button related to the specific siderail after having placed the siderail in the desired state to be monitored indicates that the monitoring in the current state is activated. Additionally, or alternatively, the indication may be provided via voice input or other means without departing from the scope of the present technology.

As an example for the present description of the method 500, monitoring the left foot siderail in the raised position will be desired. The component is therefore the left foot siderail (one of four siderails on this example hospital bed) and its desired state is raised. In this example, a single state for a single component is being monitored. As will be readily understood, multiple states and multiples components can be monitored at once by the monitoring system 400.

Once the caregiver is satisfied with the identified component(s) and state(s), they will activate monitoring by issuing a start command.

At step 501, the monitoring system 400 determines if monitoring of the component is active. Monitoring of the component will be active if the caregiver has activated monitoring at the user interface 407 as previously described. It will be appreciated that the component may include one or more components (i.e., a set of components)

An optional validation as to whether a pause command is currently active is then made at step 502. A user can proactively request that the monitoring be paused. This is typically done via the pause button 411 on the user interface 407. The pause button issues a pause command signal to the controller of the bed. The pause button 411 can be a mechanical button, a touchscreen icon or a command from an application. This proactive pause is usually commanded by the user to provide care to the patient and to avoid alerts from being issued by the monitoring system 400 during this care. The pause will typically have a predetermined duration determined by the caregiver or system administrators, for example 5 minutes. The pause is active during this duration. Once the predetermined duration has elapsed, the monitoring system 400 goes back to step 501 to determine if the monitoring of the component is still active.

In one embodiment, if, at the end of the pause, the patient is present in bed and the bed status monitoring indicates that a component is not in the desired state, an alert will be outputted. If, at the end of the pause, the patient is not present in bed, the bed enters standby mode and when a patient is next detected as present on the bed, the method 500 loops back to step 501 to determine if monitoring is active.

In one example embodiment, the pause can be stopped when the care is completed before expiry of the predetermined duration and/or can be extended if the care is not yet completed. The pause is then active for a shorter or longer duration. This is also commanded by the user using the pause button 411 which can have multiple uses, functions or sections.

In this embodiment, monitoring of the state of the component is paused if a care pause command is active. The monitoring system 400 therefore does not perform the next steps while the care pause is active. If there is a pause command active at step 502, the optional step of logging events 516 can only log that there was a pause active, optionally with a timestamp, but cannot log the potential monitoring events that occurred during the pause since the monitoring itself is paused.

Optional step 502 can be useful to manage resources and processing time by the different components of the monitoring system 400. Optional step 502 may be implemented and available in monitoring system 400 and may be called upon depending on outside conditions. For example, a power source detector may be provided on patient support apparatus 401 and may determine if the patient support apparatus 401 is connected to a power supply of the establishment or if it currently operates on its own independent battery supply. If it currently operates on battery, the monitoring system 400 may be commanded to proceed with optional step 502, while if it currently operates on the power supply of the establishment, it may be commanded to proceed directly from step 501 to step 502, skipping optional step 502. Other reasons will be apparent to those skilled in the art for implementing optional step 502, such as the availability of sufficient storage space to log all monitoring events.

If there is no active pause command or if optional step 502 is not implemented or called upon, the method 500 then proceeds to determine if the monitoring of the component is armed 503. The monitoring system 400 is not armed and does not start actively monitoring the component until the initial condition(s) for monitoring are satisfied. In particular, the monitoring will not start until the component is in the desired state. It therefore awaits the satisfaction of the initial conditions. Otherwise, it would already be in an alarm condition immediately from activation. The monitoring system 400 can be considered to be in a “preparation mode” between the activation of the monitoring and the arming of the monitoring. As soon as the initial condition is satisfied, the monitoring goes from active to armed. This can be referred to as an “auto-arm” feature, namely an automatic arming of the monitoring when initial condition(s) are satisfied.

In one embodiment, the monitoring system 400 may have an optional stabilization delay to ensure that the initial condition is satisfied and remains satisfied for more than just an instantaneous moment. For example, if a bed exit monitoring is activated and a patient is being properly placed and setup in the bed, the distribution of the weight of the patient may shift during care of the patient and the stabilization delay may ensure that in addition to a weight being detected on the bed, the position and placement of the patient is also stable and ready for monitoring.

In the example described above, the left foot siderail must therefore be raised to arm the monitoring. In some embodiments, this initial condition is sufficient and as soon as the left foot siderail is raised, the monitoring is armed. If the siderail is already raised and there is only one initial condition, then the initial condition is immediately satisfied as soon as the determination is made, and the method 500 continues.

In one embodiment, the arming of the monitoring should be done within an expected time duration. The expected time duration should give the patient and the user enough time to meet the initial condition(s). In an example embodiment, the expected time duration is 120 seconds (2 minutes). In an alternative embodiment, the expected time duration is 5 minutes.

In other embodiments, further initial conditions are to be satisfied before monitoring is armed. For example, the initial conditions may require that a patient be present on the bed before arming the monitoring system 400. This contributes to reducing alarm fatigue. The patient presence detector 403 therefore communicates an indication of its detection to the processor 431. If a patient is determined to be present on the bed, the second initial condition is satisfied. As will be readily understood, other initial conditions may be required by system administrators, by the user or by the monitoring system, depending on the required monitoring.

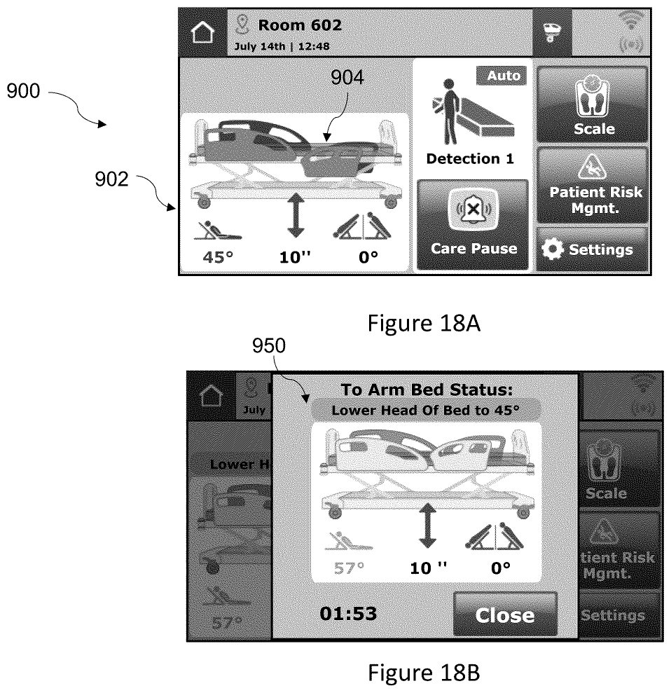

A message can be displayed on the user interface between the activation and the arming of the monitoring system 400, during the preparation mode. For example, a message can provide information to the user about what initial conditions need to be satisfied during the expected time duration. An indication of the remaining time within the expected time duration, if any, can also be shown. This contributes to a facilitated caregiver workflow. An example embodiment of this is shown in FIG. 18B.

The step 503 of verifying if initial conditions are satisfied may be thought of as a pre-monitoring step. An optional step of outputting a pre-monitoring alert indicator 504 may be executed if the monitoring of the component was not armed within the expected time duration after activation. This occurs when initial conditions are not satisfied within the expected time duration. Alert output 413 can be used to output this optional alert. This provides a safety net for caregivers and alerts them that the monitoring they thought they activated is actually not armed and will not monitor the patient. This may be helpful in a variety of circumstances and especially when a caregiver is called away to provide care to a different patient urgently and may have been distracted and forgot to meet the initial conditions for the monitoring of the patient.

Once monitoring is armed, the monitoring system 400 monitors the state of the component using sensor assembly 405 and determines if the component is in a current state different from the desired state 505. The sensor assembly 405 includes a sensor configured for providing data which enable determining what state a component is in. It therefore communicates an indication of the current state of the component to the processor 431. The processor 431, executes event detector 433 to compare the current state of the component to the desired state as inputted by the user, and determines if the component is still in the desired state or if its state is now different from the desired state. Memory 419 may store instructions related to the alarm condition for the component in alarm condition storage 423. The alarm condition may be used by the event detector 433 to make the determination of the state. Going back to the example, the sensor assembly 405 includes a sensor capable of detecting if the left foot siderail is in the raised or the lowered position. It communicates this state information to the event detector 433. If the siderail is in a lowered position, it is determined that it is in a different state than the desired position.

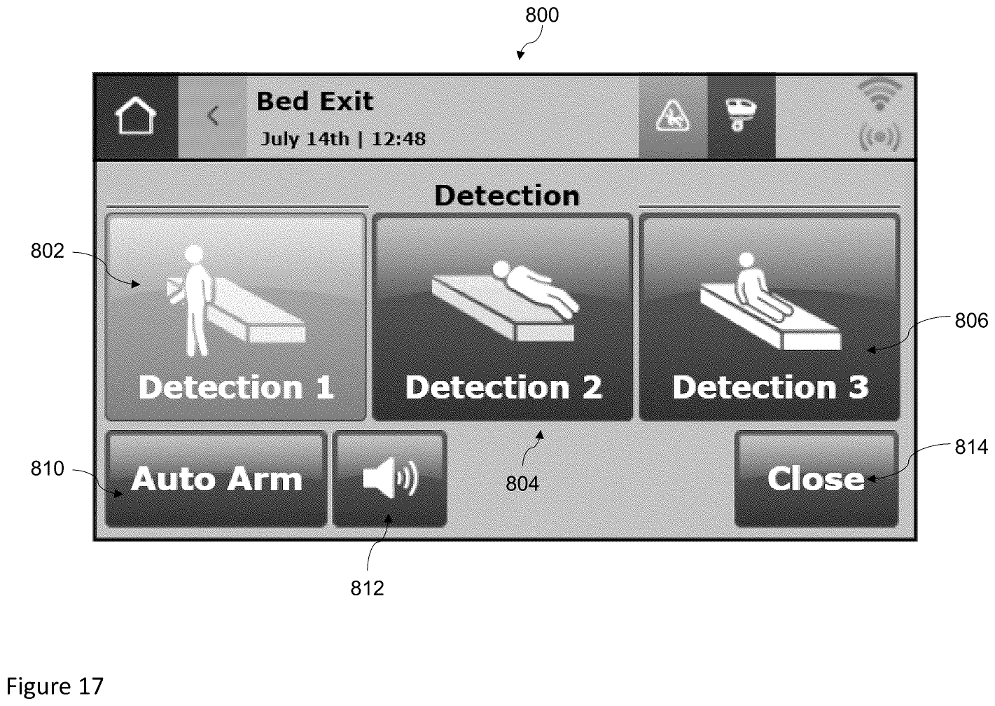

The next step is to determine if the monitoring of the component is related to bed status or bed exit at step 507. Bed exit is a special type of bed status monitoring where the monitoring is used to determine if the patient has exited the bed or is about to exit the bed. Typically, levels of bed exit monitoring are available. For example, level 3 is for monitoring if the patient has lifted their head and upper torso from the deck, level 2 is for monitoring if the patient has moved to the side of the deck and level 1 is for monitoring if the patient is no longer detected on the bed and has therefore exited the bed, as shown in the bed exit monitoring page 800 of FIG. 17, which be explained in more detail hereinafter. The component to monitor is therefore the deck of the patient support apparatus and the state is the presence/absence of a weight on the deck or the distribution of the weight on the deck or the change in the position of the detected weight (e.g., position of the center of mass within geometrical boundaries on the patient receiving surface). Prior art bed exit systems are used for this purpose. A description of an example implementation of a bed exit monitoring system is found in U.S. Pat. No. 10,497,247 by the same Applicant, the entirety of which is hereby incorporated by reference.

Memory 419 may store instructions related to the type of monitoring (bed exit or bed status) for the component and the state of the component in alarm condition storage 423. Event generator 433 then uses this type of monitoring information to make its determination.

If the armed monitoring concerns a bed exit and the component is in alarm state, the next step is to determine if a proactive care pause command is active 513. This step is similar to step 502. However, in this case, the user proactively requests that the issuance of alerts be paused but not necessarily the monitoring itself. Indeed, in this embodiment of the method 500, the determination of whether a proactive care pause is active 513 occurs after the component has been identified as being in a different state than desired state 505. The pause therefore only prevents the issuance of alerts and does not prevent the identification of an alarm condition by the event generator 433.

If an indication of a proactive care pause has not been received, the next step is the output of an alert indicator 515. This can be done via alert output 413 on user interface 407. Light assembly 415 and audio output assembly 417 on patient support apparatus 401 or on monitoring applications can also be used as needed.

An optional step of logging information about the event 516 can be carried out by the processor 431 in collaboration with the event log 425 of the memory 429. In this embodiment of the method 500, the determination of whether a proactive care pause is active 513 occurs after the component has been identified as being in a different state than desired at step 505. The event log 425 can therefore optionally log information related to the state of the component even if the alarm condition occurred during a care pause. This is useful for keeping a trace of all events that occurred while the monitoring system 400 was active, regardless of whether they occurred during a care pause. The logs are typically timestamped and include relevant information about the event.

As will be readily understood, other events can also be logged, such as the activation of the monitoring of the component at step 501 and its parameters (component, state), the arming of the monitoring system 400 at step 503 and its initial conditions, the monitoring type (bed status or bed exit), the alarm condition, etc.

If the armed monitoring type concerns a bed status other than bed exit as determined in step 507, the next step is the determination of whether the patient has left the bed at step 509. In bed status monitoring other than bed exit, the presence of the patient on the bed is usually a prerequisite for the alert to be relevant to the user. Therefore, in order to reduce alarm fatigue, the monitoring system 400 performs a verification to determine if the patient is still on the patient support apparatus. If the patient has left the bed, the bed status monitoring is considered to no longer require output of an alert indicator and therefore the system simply returns to step 501 to determine if the monitoring of the component is still active.

If the armed monitoring concerns a bed status, the component is in alarm state and the patient is still on the bed, then the next step is to determine if a proactive care pause command is active at step 513, as described above. If an indication of a proactive care pause has not been received, the next step is the output of an alert indicator 515. Again, an optional step of logging information about the event 516 in the event log 425 can be carried out.

The monitoring information collected in the event log 425 can be outputted to a user. For example, it can be printed in a printed report, displayed on a screen disposed on the patient support apparatus (e.g., the hospital bed 100), or via a remote application (app) that could optionally be accessed on a desktop or remote devices or exported in a computer-readable file.

In one embodiment, if bed status monitoring is armed and a component is placed in a different state than desired, the alert indicator will be outputted if the patient is in bed. If the patient then leaves the bed, the alert will continue to be indicated. In another embodiment, if no bed exit monitoring is armed, the alert will stop being indicated once the patient leaves the bed.

As will be readily understood, step 513 for determining if a pause command is active could be carried out earlier in the method 500 and/or repeated throughout the method 500. This would allow to pause monitoring at different points of the method 500.

When multiple components and states are being monitored at once, a priority may be assigned by the caregiver or by the system administrator. For example, bed exit monitoring may have priority over bed status monitoring. In a specific example, if both bed exit and bed status monitoring are armed and the bed status monitoring is for a raised siderail, a siderail alert indicator will be outputted if the siderail is lowered. If the patient then exits the bed, depending on the implementation, the siderail alert indicator may be stopped. However, a bed exit alert indicator will be outputted regardless.

As will be readily understood, one of the initial conditions to be satisfied may be related to the identification of a recognized caregiver. For example, a caregiver may need to enter a personal identification number (PIN) on the user interface to access the monitoring system 400 menus, to activate monitoring, to arm monitoring, to command a care pause, to stop monitoring and/or to silence an alert. The patient support apparatus may need to communicate with an internal or external database to verify that this caregiver is recognized and authorized to carry out monitoring activities. Alternatively, the caregiver may wear a personal identification device that is detected by the patient support apparatus or another device connected to the establishment network and used to detect the presence of the caregiver in the vicinity of the patient support apparatus. This would allow the logging of events to include an identification of the caregiver present with the patient and/or an identification of the caregiver who handled care for the patient. The wearable personal identification device may be a tag, card, a bracelet, a computing device, etc. The detection of the presence of the caregiver could also be used, in another embodiment, to output an alert indicator if the caregiver is determined to be absent from the vicinity of the bed after the monitoring system has been activated but before it is armed.

FIG. 11 is a photograph of a patient support apparatus 572 equipped with a patient support surface 574 in a patient room environment 570.

FIG. 12 is a photograph of a patient 576 installed on a patient support apparatus 572 equipped with a patient support surface 574 and linens in a patient room environment.

FIG. 13 is a graphical representation of a membrane which is part of control panel 600 and touchscreen 630 in one embodiment of a user interface for an endboard of the patient support apparatus. Physical buttons on the left-hand side 602 allow to control the bed actuators to reach different positions and configurations for the bed using raise buttons 606 and lower buttons 608 (not separately numbered). The physicals buttons also include locking buttons 604 (not separately numbered) to lock the current position of the different bed configurations. The touchscreen 630 on the right-hand side allows to access different pages and menus to control operation of the bed and of the monitoring system, as explained hereinafter. A battery and plug icon 634 on the right-hand side indicates how the bed is currently powered.

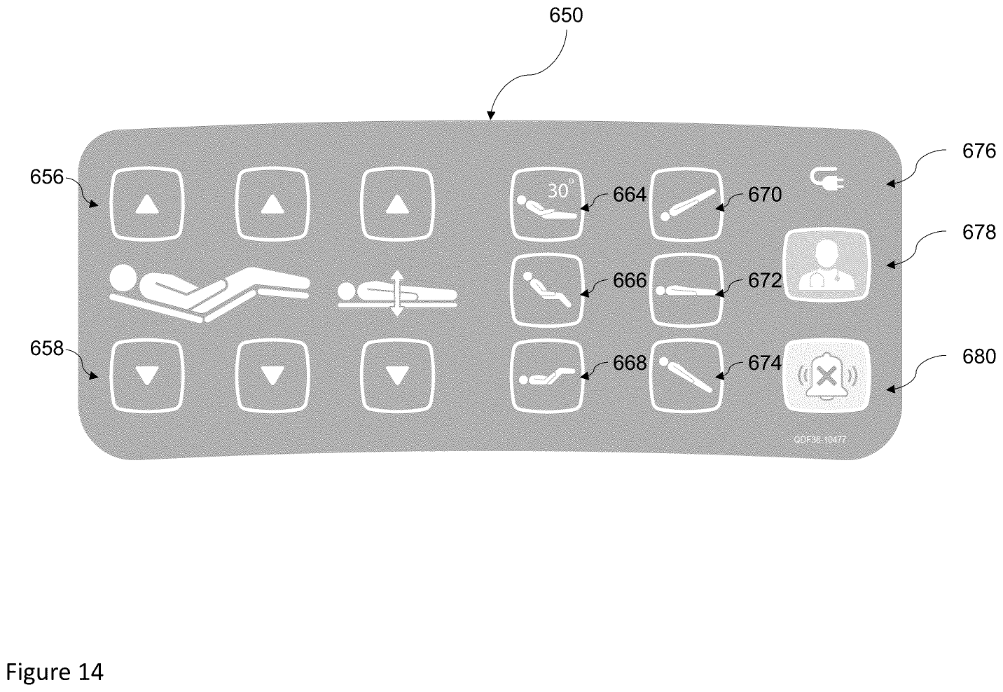

FIG. 14 is a graphical representation of a membrane which is part of control panel 650 in one embodiment of a user interface for a siderail. Similar to membrane 600, physical buttons allow to control the bed actuators to reach different positions and configurations for the bed using raise buttons 656 and lower buttons 658 (not separately numbered). Physical buttons in the middle include a 30 degree HOB position button 664, a cardiac chair position button 666, a feet raised position button 668, a Trendelenburg position button 670, a flat position button 672 and a reverse Trendelenburg position 674. There is no screen or touchscreen on this membrane to setup the monitoring system 400, but a care pause button 680 is made available (yellow bell button with an X on the bottom right hand corner). This allows the caregiver to pause monitoring directly from a side of the bed without having to walk to the endboard and facilitate the care workflow. A power icon 676 shows if the bed is plugged and nurse call button 678 are also shown.

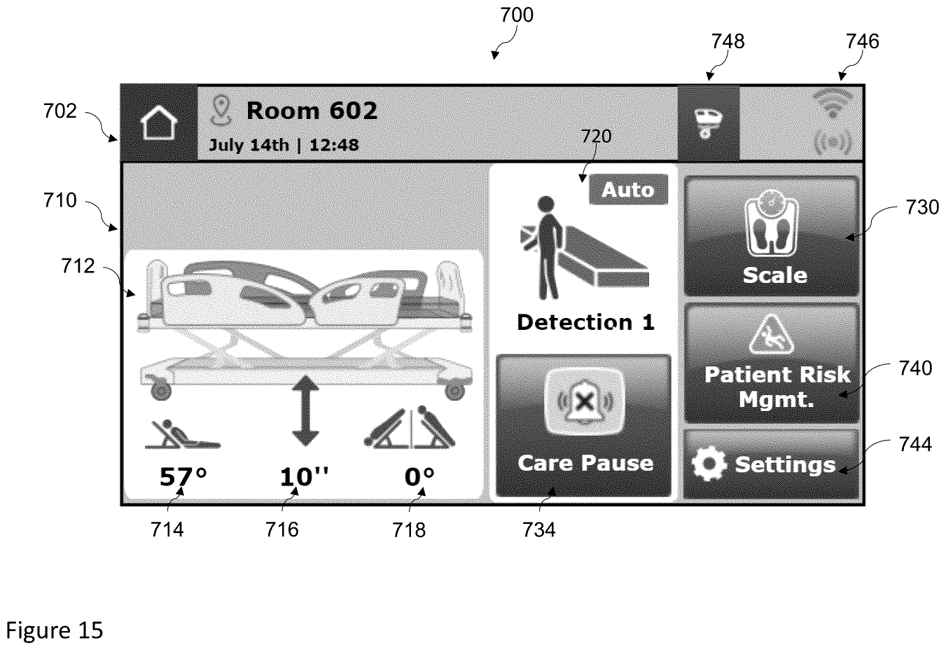

FIG. 15 is a screenshot of a home page for a touchscreen in an example embodiment of a user interface. There is shown an example of a home interface GUI 700 which may be displayed on a display device. The home GUI 700 enables inter alia to display different information related to the status and control functionalities of the bed.

The home GUI 700 includes a bed information section 710, a bed exit button 720, a scale button 730, a care pause button 734, a patient risk management button 740 and settings button 744.

The home interface GUI 700 also includes quick navigation buttons such as a home button 702 to access the home interface GUI 700, a bed status quick link button 748 to access the bed status details (FIG. 16), and a connection status icon 746, which shows if the bed is connected to a communication network and/or to a location system.

The bed information section 710 shows a graphical representation of the bed 712 with the current configuration of the bed status of the components (e.g., siderails, brakes, panels, elevation system), with a backrest icon 714 depicting the Head-of-Bed (HOB) angle with the associated value (in this example, 57 degrees), a height icon 716 indicating a height of the bed with the associated height value (in this example, 10″), and a head/foot tilt icon 718 with a value indicating an angular inclination of the bed in the Trendelenburg or reverse Trendelenburg positions (in this example, 0 degrees).

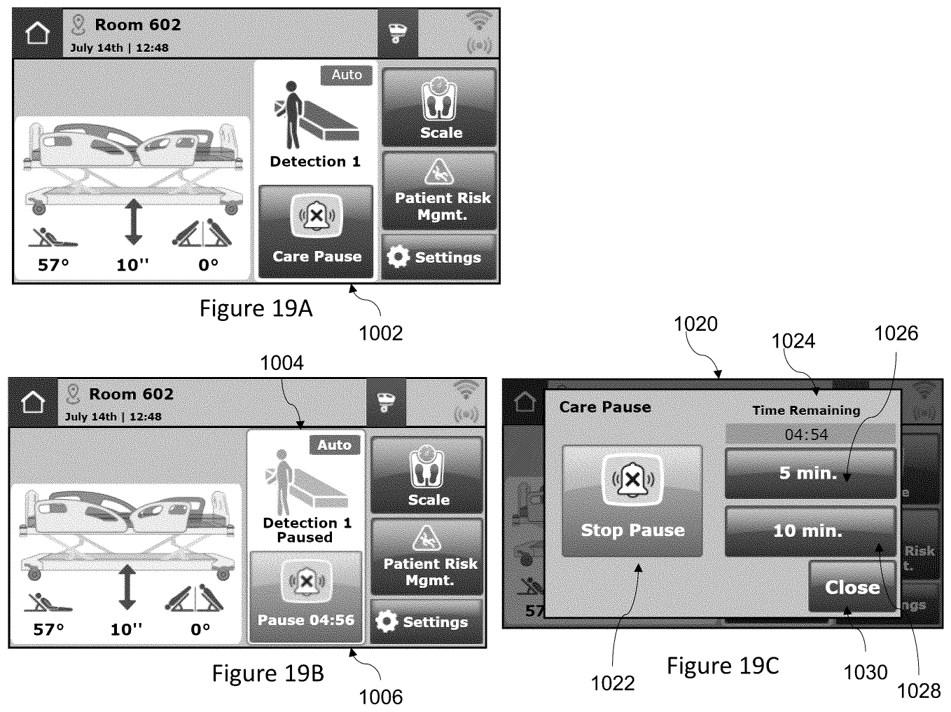

The user may click or select the bed exit button 720, the scale button 730, the care pause button 734, the patient risk management button 740 and the settings button 744 to display respectively a bed exit GUI (FIG. 17), a care pause GUI (FIG. 19), a scale GUI (not shown), a patient risk management GUI (not shown) and a settings GUI (not shown). Only those features that differ from the previously described figures are illustrated and discussed with respect to the following figures, and features that are the same are not redundantly described.

FIG. 16 is a screenshot of a bed status monitoring page 750 for activating bed status monitoring in an example embodiment. Multiple components are shown and their desired state can be indicated. Colors and values are used on the graphical representation of the bed in middle section 760 and on the component icons to highlight what components and what states are being monitored. On the left side, the bed status monitoring page 750 includes a brake status button 752, a bed height button 754, a HOB angle button 756, and a sound button 758. On the right, there is provided a right head siderail button 762, a right foot siderail button 764, a left head siderail button 766 and a left foot siderail button 768. Clicking on buttons 752, 754, 756, 762, 764, 766, 768 enables to selectively activate and deactivate the monitoring of the respective component. The start button 772 allows to activate monitoring by issuing a start command. The close button 774 enables to close the bed status monitoring page 750 and return to home GUI 700 of FIG. 15.

FIG. 17 is a screenshot of a bed exit monitoring page 800 for activating bed exit monitoring in an example embodiment. Three possible levels of detection 802, 804, 806 are made available and the auto-arm button 810 allows to activate monitoring. The close button 814 enables to close the bed exit monitoring page 800 and return to home GUI 700 of FIG. 15.

FIG. 18A shows a screenshot of a page 900 shown during preparation mode. Active bed status components 904 (side rails) which are not yet armed are highlighted in Blue. This helps to visualize what will be armed when the patient is detected as present on the bed. FIG. 18B shows a screenshot of a message 950 shown during preparation mode. The patient has been detected as present on the bed. However, an initial condition is not satisfied. The message 950 is shown to guide the caregiver in adjusting the state of the component to meet the initial condition. A timer is also started for an expected time duration. In this example, the message 950 indicates that the head of the bed should be lowered to 45 degrees, and it is currently at 57 degrees. The message 950 also indicates that 1:53 minutes are left in the preparation or stabilization mode. Additionally, lights on the bed itself may be commanded to illuminate with light of a specific color or blinking frequency to indicate that the preparation mode is ongoing.

FIG. 19A shows a screenshot of a home page where a Care Pause button 1002 is displayed. FIG. 19B shows a screenshot of a home page when a Care Pause has been commanded via the care pause button 1002 of FIG. 19A, with the bed exit status button 1004 indicating that bed exit detection is paused with the care pause button 1006 displaying the remaining time. FIG. 19C shows a screenshot of a Care Pause menu 1020 in an example embodiment. The care pause pauses all monitoring, including bed status and bed exit monitoring with the remaining time 1024 displayed (04:54 in this example). In the care pause menu, it is possible to stop the pause by pressing stop pause button 1022 or extend it by pressing the 5 min 1026 button or the 10 min button 1028. A close button 1030 enables closing the care pause menu 1020.

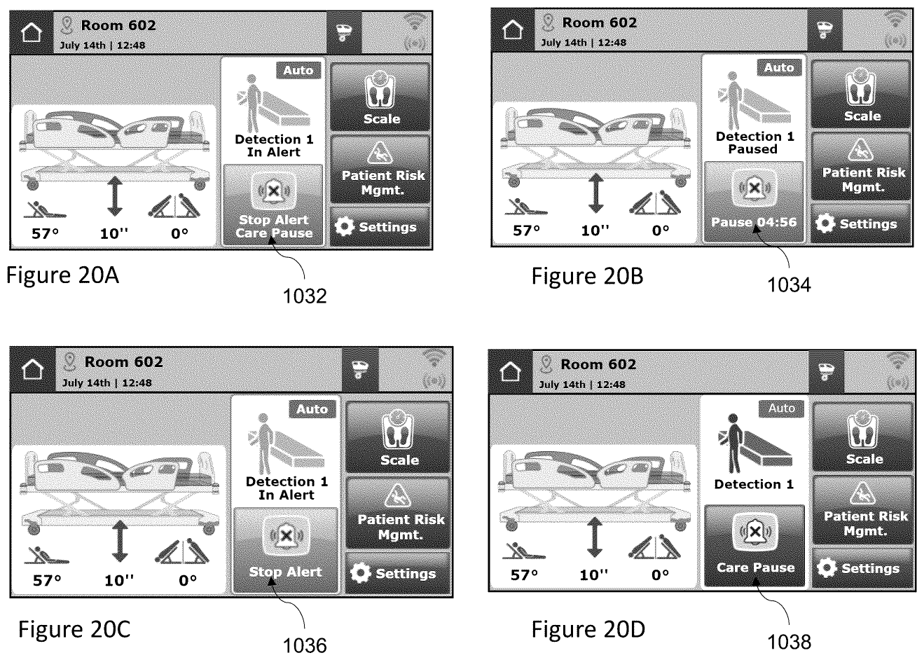

FIG. 20A shows a screenshot of a home page where a Stop Alert/Care Pause button 1032 is displayed when a patient is present in the bed. FIG. 20B shows a screenshot of a home page when a Stop Alert/Care Pause has been commanded using the stop alert/care pause button 1032, with the button 1034 now indicating the pause status and the remaining time. FIG. 20C shows a screenshot of a home page where a Stop Alert button 1036 is displayed when a patient is not present in bed. FIG. 20D shows a screenshot of a home page when a Stop Alert has been commanded by pressing stop alert button 1036, which becomes a care pause button 1038.

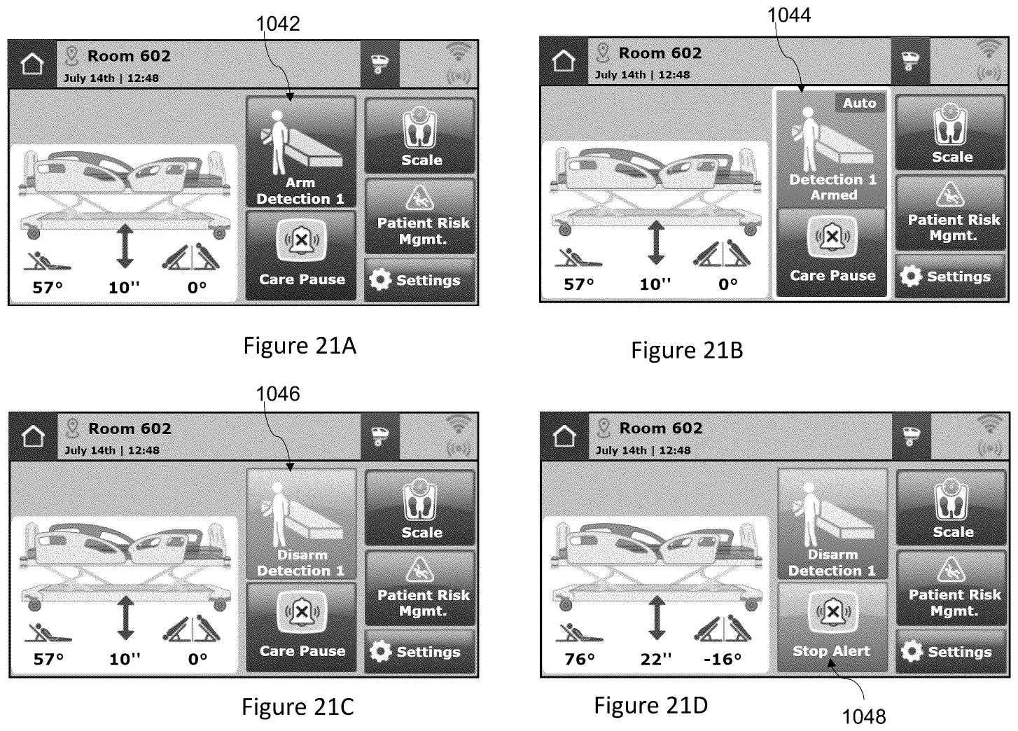

FIG. 21A shows a screenshot of a home page when the bed exit monitoring is disarmed with the bed exit monitoring button 1042 in dark color. FIG. 21B shows a screenshot of a home page where bed exit monitoring is armed with auto-arm as indicated on bed exit monitoring button 1044, which is also in lighter color. FIG. 21C shows a screenshot of a home page where bed exit monitoring is armed as indicated on bed exit monitoring button 1046. FIG. 21D shows a screenshot of a home page when a Stop Alert button 1048 is displayed because the bed exit is in alert.

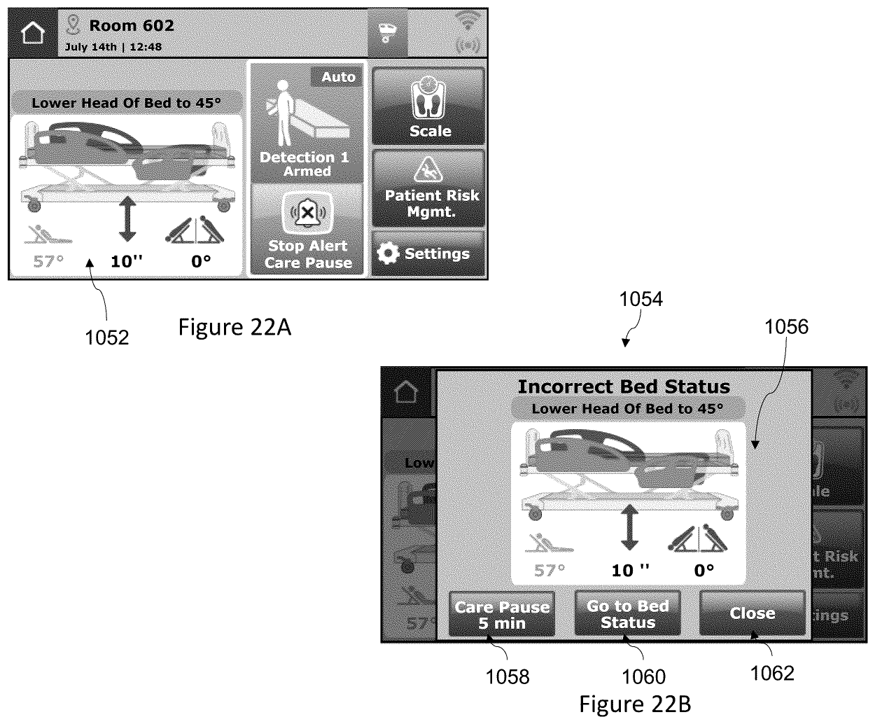

FIG. 22A shows a screenshot of a home page where there is a bed status alert as indicated on the bed status portion 1052. In order for bed status to be in an alarm condition, the bed status monitoring must be armed, and the state of the component must be different from the desired state. In this example, the desired state for the head-of-bed angle is 45 degrees but the current state is 57 degrees. FIG. 22B shows a screenshot of a detail screen showing the cause of the alert in incorrect bed status window 1054. A message 1056 is displayed to guide the caregiver in correcting the situation. The message 1056 indicates to lower the head of bed to 45 degrees and provides a shortcut care pause button 1058 for a Care pause. Four options are available: the caregiver corrects the head of bed angle and the alert indicator is stopped, the shortcut care pause button 1058 is selected by the caregiver, for example if the caregiver needs to perform some care with the patient before being able to move the head of bed, the caregiver goes to the bed status menu via go to bed status button 1060 to stop monitoring (deactivate monitoring) or the caregiver closes the pop-up window via close button 1062 and returns to the home screen.

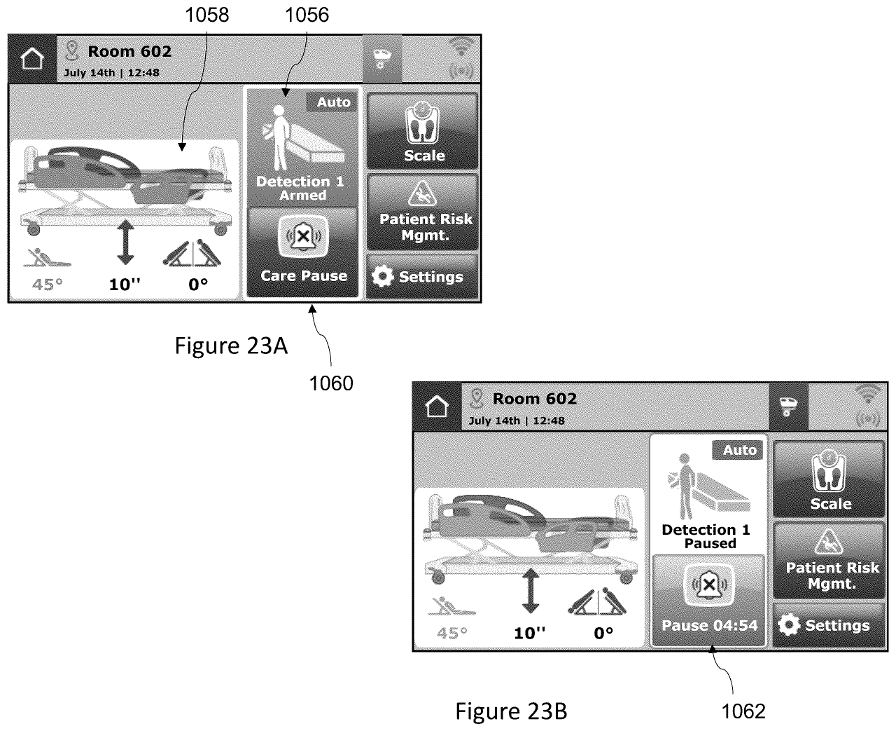

FIG. 23A shows a screenshot of a home page where bed status and bed exit are armed together when the initial condition(s) is/are satisfied as indicated by bed exit button 1066 showing that bed exit is armed at detection level 1 and the care pause button 1060 being active. FIG. 23B shows a screenshot of a home page where a Care Pause has been commanded via the Care Pause button 1060 and is in effect for both the bed exit and the bed status monitoring with care pause button 1062 showing time remaining in care pause.

FIG. 24 is a screenshot of a status board GUI 1100 for a monitoring application in an example embodiment. The application may be accessed remotely by staff via a computing device. A plurality of beds numbered 701 to 715 are monitored. Beds for which monitoring is armed and not in alarm condition are shown highlighted with a first color (beds 703, 711, 713, 714 in this example), beds for which monitoring is armed and in alarm condition are shown highlighted with a second color (bed 706 in this example). In this example, the first color is green, and the second color is red.

The components 1104 for which bed status is armed are shown highlighted on the graphical representation of the bed. The level of detection for bed exit for beds for which a bed exit monitoring is armed is shown in a section of the display. In this example, a rectangular 1106 section next to the graphical representation of the bed concerns the level of detection for bed exit. A top view of a graphical representation of the bed 1108 is used to show a center of mass of the patient on the bed. This allows to know at first glance if a patient is present on the bed. Other icons can be used to show specific conditions or risks associated with the patient, such as a fall risk.

In this example, bed 706 is currently in alarm condition and the red highlighting is the alert indicator notifying the caregiver of the alarm condition.

FIG. 25 is a screenshot of a detail page 1200 for one bed of the status board of FIG. 24. In this example, the details of bed 706 are shown. The alert indicator section 1230 includes further details on this page, including the in bed/out of bed time. There is a depiction of the patient's center of mass on the bed. The bed status monitoring section 1210 for the siderails and the head of bed angle are shown as active and not in any alarm condition. The bed exit monitoring section 1220 is shown in alarm condition. However, the patient can be seen in bed. What the user can conclude is that at some point in the past, the bed exit alarm for this patient was triggered because the patient exited the bed, and they have since come back to the bed. Looking at the in bed/out of bed graph, we can conclude that the bed exit event occurred at least 4h37 ago. If the patient was currently out of bed, the bed status would be in standby mode and the representation of the monitored components could be colored in another color, such as blue, for example.

Other states and information can also be presented on the monitoring application. For example, when monitoring is active but a care pause has been commanded, this can be shown on the monitoring application. Additionally, when a caregiver has silenced an alert, this can be shown on the application. Finally, when the patient is out of bed, this can be shown more specifically on the application. Furthermore, any information about an event that can be recorded or logged can also be displayed or represented on the application.

In one or more embodiments, method 500 and system 400 allow the following features:

The verification that initial conditions are satisfied before arming the monitoring prevents the monitoring system from detecting an alarm condition as soon as the monitoring is activated if the component is not in the desired state. This contributes to reducing alarm fatigue because it avoids output of alert indicators immediately at setup of the monitoring when the caregiver has not yet finished caring for the patient. It also allows the caregiver to setup the monitoring parameters when is most convenient for them without having to wait until the care is completed. The separation of the activation of the monitoring and the arming of the monitoring for all types of bed status monitoring and bed exit monitoring allows an automated arming of the monitoring regardless of whether the caregiver is still caring for the patient, as long as the initial conditions are satisfied.

Bed status alerts, not related to bed exit monitoring, are not indicated to the user if the patient has left the patient support apparatus. Bed status alerts other than bed exit monitoring are usually only relevant for the patient's health and safety if the patient is present on the bed. Preventing indication of these alerts when there is no patient contributes to reducing alarm fatigue as these are non-actionable alerts. If the alert indicator was outputted before the patient exited the bed, the alert indicator will continue to be active even if the patient later exits the bed.

A proactive care pause can be requested by the user after monitoring of a component is active, regardless of whether the monitoring of the component is related to a bed exit monitoring or another bed status monitoring. This contributes to reducing alarm fatigue as caregivers can proactively prevent alert indicators from being outputted while they are caring for the patient without fearing forgetting to reactivate the monitoring once they leave the patient.

The proactive care pause is different from stopping, muting, ignoring or discarding outputted alerts. Indeed, it may be possible to acknowledge an outputted alert and to request not to be reminded about this alert for a predetermined time duration using, for example, a silence button. In this case, the alert indicator was outputted by the monitoring system 400 and the user has to determine if the outputted alert indicator is relevant for the patient's health or safety or to respect establishment protocols and then explicitly interact with the outputted alert indicator to mute, postpone, ignore or discard the alert indicator. This contributes to alarm fatigue as the user receives the alert indicator and must determine what to do with the alert indicator. With the proactive care pause, the user proactively disables outputting of alert indicators during the care pause and therefore does not have to determine, for each raised alarm condition, what should be done with the alert indicator. This is especially useful when the caregiver expects to be caring for the patient for some period of time, adjusting or using the components of the patient support apparatus during this care, potentially placing them temporarily in non-desired states that would trigger alarm conditions. This therefore contributes to reducing alarm fatigue.

The present method and system allow for a single interaction with the monitoring system 400 at the beginning of a care for a patient. A single Care Pause action allows to pause all alert indicators (bed exit and bed status). At the end of the Care Pause, the outputting of alert indicators is automatically restarted, without further caregiver involvement. This reduces the mental load of the caregiver and alarm fatigue.

The preparation mode between the activation of the monitoring and the arming of the monitoring contributes to reducing false alarms and alarm fatigue as the caregiver has time to take care of placing the components in the proper states before the monitoring is armed.

While illustrated in the block diagrams as groups of discrete components communicating with each other via distinct data signal connections, it will be understood by those skilled in the art that the illustrated embodiments may be provided by a combination of hardware and software components, with some components being implemented by a given function or operation of a hardware or software system, and many of the data paths illustrated being implemented by data communication within a computer application or operating system. The structure illustrated is thus provided for efficiency of teaching the described embodiment.

Modifications and improvements to the above-described embodiments of the present technology may become apparent to those skilled in the art. The foregoing description is intended to be exemplary rather than limiting. The scope of the present technology is therefore intended to be limited solely by the scope of the appended claims.

1. A monitoring system for a patient support apparatus, the monitoring system comprising:
a sensor assembly operatively connected to the patient support apparatus, the sensor assembly being operable to provide an indication of a state of a component of the patient support apparatus;
a user interface operable to receive at least one input and provide at least one output,
the input being indicative of at least one of: a start command to activate monitoring of the component and a pause command to pause outputting an alert indicator for the activated monitoring of the component,
the output being indicative of the alert indicator;
a memory operable to store computer-readable instructions including instructions indicative of an alarm condition for the state of the component; and
a processing unit operatively connected to the sensor assembly, the user interface and the memory, the processing unit being operable to:
receive, from the user interface, the at least one input;
determine if at least one initial condition has occurred, wherein the at least one initial condition includes a presence of the patient on the patient support apparatus;
determine if the alarm condition has occurred based on the input and the state of the component; and
provide the alert indicator if the alarm condition has occurred, in the presence of the initial condition, in the presence of the start command and in an absence of the pause command.

2. The monitoring system as claimed in claim 1, wherein the processing unit is operable to:
determine if the state of the component is related to one of a bed exit of the patient and a bed status of the patient support apparatus;
determine if the patient is currently present on the patient support apparatus; and
provide the alert indicator if the state of the component is related to the bed status and if the patient is currently present on the patient support apparatus.

3. The monitoring system as claimed in claim 1, further comprising at least one of: a light assembly, an audio output assembly, a display and an application operatively connected to the processing unit to provide the alert indicator.

4. The monitoring system as claimed in claim 3, wherein the user interface comprises at least one of: a touchscreen and a control element configured to receive the input from the user.

5. The monitoring system as claimed in claim 1, wherein the memory comprises storage for a log; and wherein the processing unit is further operable to log at least one of: an indication of an occurrence of the start command, an indication of an occurrence of the pause command and an indication of the determination that the alarm condition has occurred.

6. The monitoring system as claimed in claim 1, wherein the input is indicative of a desired state for the component and wherein the processing unit is operable to use the desired state for the determining if the alarm condition has occurred.

7. The monitoring system as claimed in claim 6, wherein the component comprises at least one of:
a siderail wherein the desired state is the siderail being raised,
a brake wherein the desired state is the brake being set,
an elevation system wherein the desired state is one of a height of the patient support apparatus and an angle of a tilt of the patient support apparatus, and
a panel wherein in the desired state is an angle of the panel.

8. The monitoring system as claimed in claim 1, wherein the sensor assembly comprises at least one load cell configured for determining at least one of a weight distribution of the patient on the patient support apparatus and a presence of the patient on the patient support apparatus.

9. The monitoring system as claimed in claim 1, wherein if the input is the pause command, the processing unit is further configured for:
displaying a time remaining in the pause on the user interface;
displaying a pause extension on the user interface to extend the pause; and
in response to the time remaining expiring and the user not selecting the pause extension:
providing the alert indicator.

10. A patient support apparatus comprising:
a frame;
a patient receiving surface supported by the frame;
a sensor assembly operable to transmit an indication of a state of a component of the patient support apparatus;
a user interface operable to receive at least one input and provide at least one output,
the input being indicative of at least one of a start command to activate monitoring of the component and a pause command to pause outputting an alert indicator for the activated monitoring of the component, and
the output being indicative of the alert indicator;
a memory operable to store computer-readable instructions including instructions indicative of an alarm condition for the state of the component; and
a processing unit operatively connected to the sensor assembly, the user interface and the memory, the processing unit being operable to:
determine if at least one initial condition has occurred, wherein the at least one initial condition includes a presence of the patient on the patient receiving surface;
determine if the alarm condition has occurred based on the input and the state of the component; and
provide the alert indicator if the alarm condition has occurred, in the presence of the initial condition, in the presence of the start command and in an absence of the pause command.

11. The patient support apparatus as claimed in claim 10, wherein the processing unit is operable to:
determine if the state of the component is related to one of a bed exit of the patient and a bed status of the patient support apparatus;
determine if the patient is currently present on the patient support apparatus; and provide the alert indicator if the state of the component is related to the bed status and if the patient is currently present on the patient support apparatus.

12. The patient support apparatus as claimed in claim 11, wherein the input is indicative of a desired state for the component and wherein the processing unit is operable to use the desired state for the determining if the alarm condition has occurred.

13. The patient support apparatus as claimed in claim 12, wherein the component comprises at least one of:
a siderail wherein the desired state is the siderail being raised,
a brake wherein the desired state is the brake being set,
an elevation system wherein the desired state is one of a height of the patient support apparatus and an angle of a tilt of the patient support apparatus, and
a panel wherein in the desired state is an angle of the panel.

14. A monitoring method for a patient support apparatus, the monitoring method being executed by at least one processing unit operatively connected to a patient support apparatus and at least one user interface for receiving at least one input and providing at least one input, the monitoring method comprising:
determining a state of a component of the patient support apparatus;
receiving at least one input, the input being indicative of at least one of a start command to activate monitoring of the component and a pause command to pause outputting an alert indicator for the activated monitoring of the component;
providing at least one output, the output being indicative of the alert indicator;
retrieving instructions indicative of an alarm condition for the state of the component;
determining if at least one initial condition has occurred, wherein the at least one initial condition includes a presence of the patient on the patient support apparatus;
determining if the alarm condition has occurred based on the input, the instructions and the state of the component; and
providing the alert indicator if the alarm condition has occurred, in the presence of the initial condition, in the presence of the start command and in an absence of the pause command.

15. The monitoring method as claimed in claim 14, further comprising
determining if the state of the component is related to one of a bed exit of the patient and a bed status of the patient support apparatus;
determining if the patient is currently present on the patient support apparatus; and
wherein the providing the alert indicator is dependent on if the state of the component is related to the bed status and if the patient is currently present on the patient support apparatus.

16. The monitoring method as claimed in claim 14, wherein the input is indicative of a desired state for the component and wherein the method comprises determining if the alarm condition has occurred based on the desired state.

17. The monitoring method as claimed in claim 16, wherein the component comprises at least one of:
a siderail wherein the desired state is the siderail being raised,
a brake wherein the desired state is the brake being set,
an elevation system wherein the desired state is one of a height of the patient support apparatus and an angle of a tilt of the patient support apparatus, and
a panel wherein in the desired state is an angle of the panel.

18. The monitoring method as claimed in claim 17, wherein if the input is the pause command, the method further comprises:
displaying a time remaining in the pause on the user interface;
displaying a pause extension on the user interface to extend the pause; and
in response to the time remaining expiring and the user not selecting the pause extension:
providing the alert indicator.

19. The monitoring method as claimed in claim 14, further comprising:
storing, in a log, at least one of: an indication of an occurrence of the start command, an indication of an occurrence of the pause command and an indication of the determination that the alarm condition has occurred.

20. The monitoring method as claimed in claim 14, wherein the user interface comprises a touch screen.
